(19) United States
(12) Patent Application Publication
(10) Pub. No.: US 2024/0299091 A1
(43) Pub. Date: Sep. 12, 2024

(54) Radial Pulse Field Ablation Catheter System

(71) Applicants: The Trustees of Dartmouth College, Hanover, NH (US); Mary Hitchcock Memorial Hospital, For Itself and On Behalf of Dartmouth-Hitchcock Clinic, Lebanon, NH (US)

(72) Inventors: Kevin F. KWAKU, Hanover, NH (US); Alexander HATTOUM, North Huntingdon, PA (US); B. Stuart TREMBLY, Hanover, NH (US)

(21) Appl. No.: 18/562,514

(22) PCT Filed: Jan. 28, 2022
(86) PCT No.: PCT/US2022/014431

§ 371 Date: Nov. 20, 2023

Related U.S. Application Data
(60) Provisional application No. 63/191,164, filed on May 20, 2021.

(51) Int. Cl. A61B 18/16; A61B 18/00; A61B 18/12; A61B 18/14

(52) U.S. Cl. CPC A61B 18/16; A61B 18/1206; A61B 18/1492; A61B 2018/00577; A61B 2018/00613; A61B 2018/1253; A61B 2018/1266; A61B 2018/165; A61B 2018/167

(57) ABSTRACT

A tissue ablation system and method are provided. The system includes a catheter including a distal electrode tip having at least one tip electrode, at least one return electrode positionable independently of the tip electrode, and a voltage generator circuit electrically connected to the tip electrode and to the return electrode. The tip electrode and the return electrode include a dielectric or other non-conductive surface layer. The voltage generator circuit applies a voltage differential across the tip electrode and the return electrode such that the tip electrode and the return electrode are given opposite polarity. Tissue located between the tip electrode and the return electrode acts as a capacitive dielectric medium in which an electric field is present and is not dependent on current flow between the tip electrode and the return electrode, and the electric field induces electroporation of tissue cells in a vicinity of the distal electrode tip.

### CROSS-REFERENCE TO RELATED APPLICATIONS

This application claims priority to the provisional patent application filed May 20, 2021 and assigned U.S. App. No. 63/191,164, the disclosure of which is hereby incorporated by reference.

### FIELD OF THE DISCLOSURE

This disclosure relates to a system and method for delivering radially directed and temporally stable electric fields for pulse field ablation (“PFA”) of pathological tissue associated with cardiac arrhythmias and other diseases (including tumors and neoplasia) via the process of irreversible electroporation (“IRE”) to induce cell death.

### BACKGROUND OF THE DISCLOSURE

There are many medical treatments that involve instances of cutting, ablating, coagulating, destroying, or otherwise changing the physiological properties of tissue. These techniques can be used beneficially to remove diseased tissue or change the electrophysiological properties of tissue, such as those associated with cardiac arrhythmias or other electrophysiological abnormalities.

Catheter ablation for cardiac arrhythmias is a major therapeutic tool in modern cardiac medicine. Currently, radiofrequency tissue heating is the predominant method of ablating cardiac arrhythmias. Although often effective, radiofrequency heating requires direct tissue contact, may indiscriminately destroy all cell types, induces inflammation, and may perforate essential structures. Recently, the method of pulse electric fields has emerged as a means of ablating pathological tissue/cells via the process of irreversible electroporation (IRE). Application of high voltage pulses to a tissue can disrupt cell membranes by generating pores. If the applied electric field at the membrane is larger than a threshold value and maintained for a stable but short period of time (e.g., approximately 50-200 microseconds), the electroporation is irreversible and the pores remain open, permitting exchange of material across the membrane and leading to non-inflammatory apoptosis (cell death) without destruction of structural tissue elements.

Electroporation can depend on the establishment of a supra-physiologic transmembrane voltage on the individual target tissue cells. Transmembrane voltages (TMVs) with a constant field may generate a direct current through tissue. This can cause side effects and can be unpredictable in complex tissue.

Previous catheter ablation systems for PFA utilized localize dipoles or provided extended dipoles by way of high current shocks. These previous systems exhibit various disadvantages.

A first disadvantage is that previous systems using localized dipoles have limited ablation efficiency. IRE ablation efficiency is proportional to the field strength perpendicular to cell membranes. In previous systems using localized dipoles, illustrated in FIGS. 1 and 2, the electric field lines are directed predominantly parallel to the spacing direction of the positive and negative dipole electrodes within the catheter tip. Thus, the electric field lines are mostly parallel to cell membranes in the local target region, which drastically limits ablation efficiency.

A second disadvantage of previous systems is their rapid decay of field intensity. One promise of field-based approaches is that fields can penetrate matter beyond the local area of the catheter tip. In treating cardiac dysrhythmias, the ability to provide ablation of aberrant conduction through a cardiac wall would greatly enhance the long term efficacy of such procedures. Indeed, a lack of transmurality is a recognized limitation of previous radiofrequency methods due to possible perforation. It is also a limitation of previous systems using localized dipoles, wherein the electric fields attenuate rapidly with distance from the source electrode on the order of about 1/r³, where “r” equals the radial distance from the source electrode. Further in regard to localized dipoles, the electric field lines are predominantly parallel to the cell membranes of the target tissue, resulting in rarer and heterogeneous ablation. This can create critical gaps in ablated tissue that may generate further dysrhythmias. For these reasons, previous systems are not suitable for targeting transmural pathologic cardiac substrate.

A third disadvantage of previous systems arises from high current delivery to the patient, which is a source of unintended thermal injury, unwanted perioperative cardiac dysrhythmia, and musculoskeletal injury from skeletal muscle stimulation. In previous PFA systems using localized dipoles, the electrodes at the tip of the catheter are closely spaced and are operated at a high voltage differential, raising the risk of thermal injury currents. For this reason, pulsed electric fields with high temporal resolution pulses (i.e. on the scale of 10 to 100 microseconds) are provided in such previous systems.

In previous systems which provide extended dipoles by way of high current shocks, such as that shown in FIGS. 3 and 4, it has been observed that the PFA electric field generation is entirely dependent on the current delivery to the patient, and may reach the equivalent of a 200 Joule defibrillation shock. The electric field matches the direction of the overall current. Although at times this can be radially directed, the electric field will immediately mirror the more unpredictable direction of current delivery. Consequently, the direction of the electric field lines in relation to the catheter is unstable, thus limiting catheter based guidance regardless the catheter tip design. Moreover, the 200 Joule shocks are defibrillatory levels of energy which limit dose delivery to single doses at a time and overall few deliveries per procedure. Finally, such high-current designs are not compatible with power delivery systems with high frequency components without harmful radiofrequency currents, and, therefore, they are not compatible with high temporal resolution systems.

A fourth disadvantage which concerns previous systems using current-based electric field delivery is stray and parasitic capacitance of drive lines to any conductive circuit components within the system or areas of non-target tissue with adequate conductive properties, whereby delivery of high voltages to catheter tip electrodes are siphoned by unanticipated fields between the high voltage electrodes in close proximity. Previous systems lack adequate protection or mitigation of this negative effect.

Thus, what is needed is a PFA system to selectively perform IRE to target tissue while minimizing damage to healthy tissue. Specifically, what is needed is a PFA system which provides improved ablation efficiency and the possibility of transmurality via radially directed, temporally stable fields that do not rely on high current flow for emission as compared to previous systems.

### BRIEF SUMMARY OF THE DISCLOSURE

An embodiment of the present disclosure provides a tissue ablation system. The system may comprise a catheter including a distal electrode tip having at least one tip electrode. The at least one tip electrode may comprise a dielectric or non-conductive surface layer. The system may further comprise at least one return electrode positionable independently of the at least one tip electrode. The at least one return electrode may comprise a dielectric or non-conductive surface layer. The system may further comprise a voltage generator circuit electrically connected to the at least one tip electrode and to the at least one return electrode. The voltage generator circuit may be operable to apply a voltage differential across the at least one tip electrode and the at least one return electrode such that the at least one tip electrode may be given a polarity opposite a polarity of the at least one return electrode. Tissue located between the at least one tip electrode and the at least one return electrode may act as a capacitive dielectric medium in which an electric field is present and is not dependent on current flow between the at least one tip electrode and the at least one return electrode, and the electric field may induce electroporation of tissue cells in a vicinity of the distal electrode tip.

According to an embodiment of the present disclosure, the at least one return electrode may have a surface area greater than a surface area of the at least one tip electrode.

According to an embodiment of the present disclosure, the at least one return electrode may be separate from the catheter. The at least one return electrode may be embodied in a patch configured for external placement on a skin surface.

According to an embodiment of the present disclosure, the voltage generator circuit may be an AC voltage generator circuit.

According to an embodiment of the present disclosure, the voltage generator circuit may be a DC voltage generator circuit.

According to an embodiment of the present disclosure, the catheter may be a steerable catheter.

According to an embodiment of the present disclosure, the electrode tip may be configured to be rotatable. The catheter can define an aperture that the electric field is emitted through.

Another embodiment of the present disclosure provides a tissue ablation method. The method may comprise positioning a first electrode at a location proximate to a tissue target. The first electrode may comprise a dielectric or non-conductive surface layer. The method may further comprise positioning a second electrode at another location spaced from first electrode and the tissue target. The second electrode may comprise a dielectric or non-conductive surface layer. The method may further comprise generating a voltage differential across the first electrode and the second electrode such that the first electrode has a positive polarity and the second electrode has a negative polarity. Tissue located between the first electrode and the second electrode may act as a capacitive dielectric medium in which an electric field is present and is not dependent on current flow between the first electrode and the second electrode, and the electric field may induce electroporation of tissue cells in the tissue target.

According to an embodiment of the present disclosure, the second electrode may have a surface area greater than a surface area of the first electrode. The electric field may be distributed across most of the surface area of the second electrode.

According to an embodiment of the present disclosure, the step of positioning the first electrode may comprise guiding the first electrode endovascularly within a patient by means of a catheter. The first electrode can be rotated thereby exposing different areas of the tissue to the electric field through an aperture. The electric field may be primarily emitted through the aperture in the catheter.

According to an embodiment of the present disclosure, the step of positioning the second electrode may comprise placing the second electrode against an external skin surface of a patient. More than one of the second electrode may be provided, and a pair of the second electrodes may be positioned at locations on opposite sides of a patient.

According to an embodiment of the present disclosure, the electric field extends in all directions from the first electrode.

According to an embodiment of the present disclosure, the electric field is a monopole with radially-directed fields from the first electrode.

According to an embodiment of the present disclosure, the electric field has radially-extending electric field geometries in all directions from the first electrode.

### DESCRIPTION OF THE DRAWINGS

For a fuller understanding of the nature and objects of the disclosure, reference should be made to the following detailed description taken in conjunction with the accompanying drawings, in which:

FIG. 1 is a schematic view illustrating an example of a localized dipole PFA catheter system known from the prior art;

FIG. 2 is a schematic view illustrating another example of a localized dipole PFA catheter system known from the prior art having a ring-shaped electrode tip;

FIG. 3 is a schematic view illustrating a shock-based high current extended dipole PFA system proposed in the prior art;

FIG. 4 is a schematic circuit diagram of the prior art extended dipole PFA system of FIG. 3 in conjunction with a patient;

FIG. 5 is a schematic view illustrating a novel PFA catheter system according to an embodiment of the present disclosure deployed in a patient during surgery;

FIG. 6 is an enlarged schematic view of an electrode tip of the novel PFA system shown in FIG. 5, illustrating directional electric field lines in the vicinity of cardiac cells;

FIG. 7 is a simplified schematic circuit diagram of the novel PFA catheter system of FIG. 5 in conjunction with a patient;

FIG. 8 is a schematic diagram illustrating a pulse generator system of the novel PFA catheter system according to an embodiment of the present disclosure;

FIG. 9 is schematic diagram of the novel PFA catheter system, illustrating possible length characteristics of conductors of the system according to an embodiment of the present disclosure;

FIG. 10 is a longitudinal cross-sectional view showing an electrode tip usable in the novel PFA catheter system according to an embodiment of the present disclosure;

FIG. 11 is a schematic perspective view showing another electrode tip usable in the novel PFA catheter system according to an embodiment of the present disclosure, wherein the electrode tip includes floating capacitive elements for guiding electric field emissions;

FIG. 12 is an electrical schematic diagram representing an embodiment of the catheter system using the electrode tip shown in FIG. 11

FIG. 13 is a schematic perspective view showing another electrode tip usable in the novel PFA catheter system according to an embodiment of the present disclosure, wherein the electrode tip has a deformable spheroid configuration with spaced electrodes;

FIG. 14 is a schematic end view of the electrode tip shown in FIG. 13;

FIG. 15 is a cross-sectional view of a return patch usable in the novel PFA catheter system according to an embodiment of the present disclosure;

FIG. 16 is a rear plan view of the return patch shown in FIG. 15;

FIGS. 17A and 17B are end views showing a conductive electrode plate of the return patch in a flat condition and a bent condition, respectively;

FIG. 18 is a schematic view illustrating use of a novel catheter tip system of the present disclosure for a non-cardiac procedure in the nature of tumor ablation for interventional oncology;

FIG. 19 is an exemplary chart of conductivity and permittivity versus frequency for heart muscle;

FIG. 20 is an exemplary chart of voltage versus frequency;

FIG. 21 shows a local monopole geometric variation with two negative pole patches at 20 cm separation;

FIG. 22 shows an extended dipole (local monopole) with computed tomography (CT) overlap;

FIG. 23 is an example showing that an extended dipole can create a local monopole with radially-directed fields from a central catheter tip;

FIG. 24 is an example with a four patch configuration;

FIG. 25 is another example with a four patch configuration;

FIG. 26 illustrates the orientation of the tip with respect to isotropy;

FIG. 27 shows a radial-directed fields simulation;

FIG. 28 shows a gap-free lesion extension;

FIG. 29 show a simulation from 1-4 microseconds, which each chart representing a 1 microsecond snapshot during this period;

FIG. 30 shows isotropy for various orientations;

FIG. 31 illustrates time-dependence relationships for embodiments disclosed herein;

FIG. 32 is a cross-sectional view of an embodiment of a rotatable electrode tip;

FIG. 33 is a schematic of a catheter embodiment using the embodiment of FIG. 32;

FIG. 34 illustrates free space simulations of electrode geometries;

FIG. 35 illustrates two-dimensional direct current pulse simulations of dipole and pseudomonopole geometries in varying tissue tip orientations;

FIG. 36 illustrates direct current pulse single dose ablation threshold volumes;

FIG. 37 illustrates on-face and tilted direct current single dose ablation threshold volumes;

FIG. 38 illustrates high frequency based sinusoidal (electroquasistatic H-FIRE) energy delivery simulations of pseudomonopole geometry using 0.03 mm conductive graphite tip coating;

FIG. 39 illustrates enhanced current reduction pseudomonopole high frequency (H-FIRE) simulations with high resistance 0.03 mm tip coatings;

FIG. 40 illustrates a single dose ablation threshold volume comparisons between energy delivery methods; and

FIG. 41 illustrates a tissue tip extensions effect.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure. Accordingly, the scope of the disclosure is defined only by reference to the appended claims.

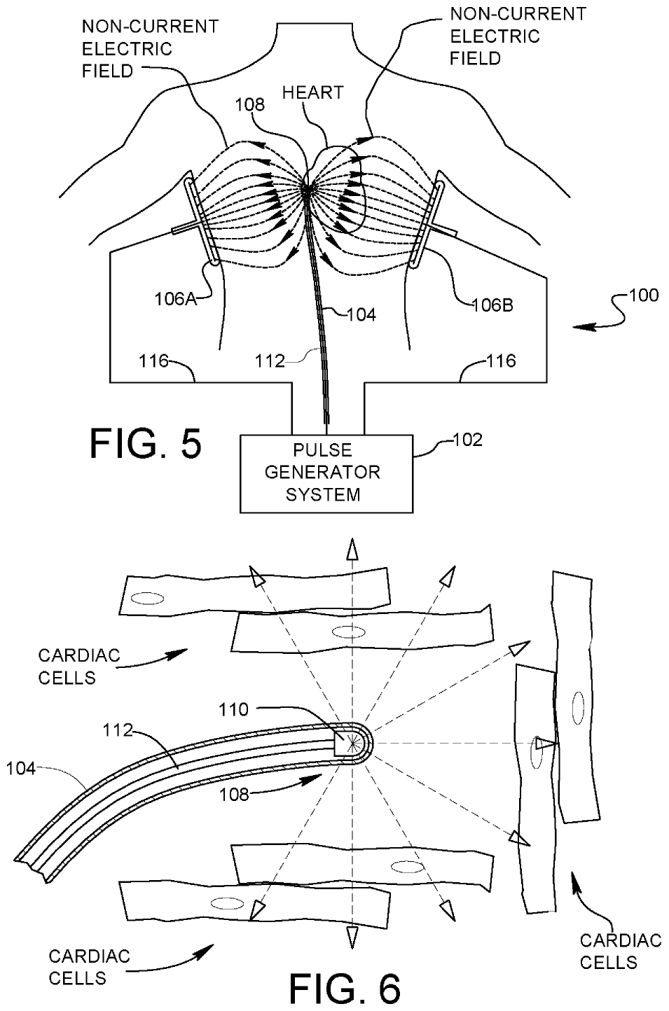

FIGS. 5-9 illustrate a novel PFA catheter system 100 according to an embodiment of the present disclosure. System 100 generally comprises components configured to deliver radially directed and transmural electric fields for surgical PFA of tissue. For example, system 100 may be used to ablate pathological tissue associated with cardiac arrhythmias and other diseases, including various types of cancer. The PFA catheter system 100 can deliver sustained IRE-TMVs for pulse field ablation in tissue, such as cardiac tissue.

Electroporation can depend on the establishment of a supra-physiologic transmembrane potential on the individual target tissue cells. The radially-directed electric field can achieve IRE-capable TMVs on target tissue cells. In the embodiments disclosed herein, the field is produced in a manner that can minimize harmful and unpredictable large pulses of direct current. Embodiments disclosed herein can enhance operator ease of use, can improve on lesion formation in a transmural fashion, and can create more stable and predictable field distributions without relying solely on high amperage current.

The disclosed system, which can use an extended dipole, can create radially-extending electric field geometries in all directions from the catheter tip. The tip patch extended dipole system can concentrate a high-intensity field at the tip for targeted ablation while attenuating return field energy to non-ablative strengths at the patches. An invariance of effective field to tip orientation (isotropy) can assist an operator.

Radial fields can allow the ablated/non-ablated tissue interface to extend the effect of the tip. Tissue ablated from a dose of electric field can become pore laden and, thus, more conductive. Additional doses of applied field can create iterative depth if the direction of the applied field directs tissue ions toward the interface (e.g., radial-directed). Radial fields that are applied iteratively can create lesions that grow in continuous fashion without gaps. An electroquasistatic (EQS) method of field delivery can limit conductive direct harmful currents by allowing dielectric re-enforced coatings and enabling waveforms that provide fully biphasic dosages of field.

While not intending to be limited to the mechanics and explanation provided, electroporation can be understood in terms of a chemical equilibrium problem. Cell membranes form both hydrophilic and hydrophobic pores via thermal fluctuations. Pore density can be dictated by thermodynamic energy based distributions. At baseline, the energy tends to favor sealed membranes. These equilibriums can be pushed toward the formation of more and larger hydrophilic pores by increasing TMV. When a cell is exposed to a stable electric field, a supra-physiologic TMV can be induced by this field. Under a sufficiently-elevated TMV, hydrophilic pores become energetically favorable. Under the strain of a TMV, the membrane becomes a capacitor separating charges resisting the flow of ions. By evaluating the free energy of formation of a pore membrane, a hydrophilic pore can reduce the membrane energy by a factor of a discharged capacitor. Typical of a discharging capacitor, some or all of the energy reduction can be proportional to the square of the TMV. Potential energy landscapes can be used to characterize the favorability of pore formation.

At various TMVs, a local minimum is seen with a spontaneous pore size even at a baseline. Critical pore lengths beyond this energy continue to minimize at increasing pore radii, which is associated with irreversibility.

Regarding a kinetic analysis of pore formation rates, a thermodynamic scheme for membrane and cellular processes can be applied. Often the slowest step can dictate the rate of the entire process in chemical kinetics (i.e., a rate limiting step). Within a viscous membrane, a diffusion limited kinetic model can be derived whereby the slowest aspect of pore density production at a point is limited by the ability of pore boundaries to diffuse through a viscous membrane. The rate of diffusion can be dictated by random thermal fluctuations, the viscosity of the medium, and/or any biasing force dictated by reducing the energy of formation. Quantifying this rate of biased diffusion of the pore edge may characterize the kinetics. In an instance, the Smoluchowski equation can be simplified to give a relationship between TMV at a point on a membrane and total pore density (of any radius) at that corresponding point.

The relationship between an applied electric field generated from a catheter system and the TMV that field induces at the cellular level can be used to design a PFA system with optimized efficiencies. In an idealized system, a spherical bilayer exposed to a uniform electric field E (uniform over the scale of the cell size). The Schwan equation can be derived to relate the transmembrane voltage at a point on this membrane, as shown below.

$$TMV = 3 / 2 r ‖ E ‖ cos θ$$

Here r is the distance from the cell center and θ is the angle formed between the electric field and the line perpendicular to that point on the membrane. The TMV may be maximized if the applied field is perpendicular to the membrane. This model of a simple spherical single membrane can be expanded to more complicated shapes and multiple membrane cells. The perpendicularity principle typically remains for an applied field. In cardiac tissue histology, the cells approximate elongated circular fibers that align in parallel to a blood tissue interface.

A time-varying electric field also can be applied. The overall form of Schwan's equation again can be applied. A quasistatic approximation would typically be applicable on the length scales for these applications up to high frequencies with a non-linear frequency dependency factor.

Perpendicularity and field strength may scale to pore rates as e^{(TMV}^{²}⁾. Thus, maximizing perpendicularity can increase rates without a need to increase in direct current or applied voltages. A radial isotropic emission pattern can maximize perpendicular orientations of field to the long axis of cardiac cells from endocardiac catheter especially if various targets on the cardiac surface are required.

In direct current pulse methods, field strength will be based on direct current. Side effects and tissue damage will relate to the product of current and time. Ablation rates can scale to field strength as e^{(TMV}^{²}⁾, while scaling linearly to the pulse time. Therefore, asymmetric time pulses can reduce times at high direct currents. Frequency-based methods can induce TMV with combination of both direct current and displacement currents.

From a clinical perspective, efficiency can include increasing ablation depth where needed while reducing direct current and not generating arrhythmogenic ablation gaps. In actual cases, the ability to deliver the catheter to a specific target can vary from patient to patient. Using a specific orientation of the catheter tip to the tissue/blood interface can increase the difficulty of the procedure and reduce uniformity amongst operators. These issues are addressed using the embodiments disclosed herein.

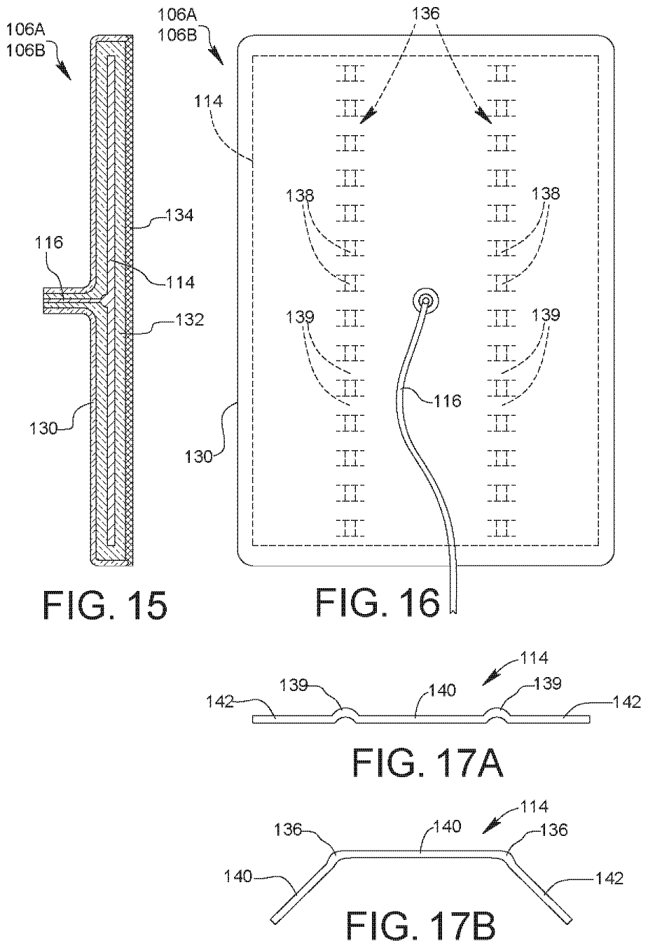

PFA catheter system 100 may comprise a pulse generator system 102 of either direct current or alternating current type, a steerable catheter 104, and one or more return patches 106A and 106B. While two return patches 106A and 106B are illustrated, more than two return patches (e.g., four return patches) can be used. Catheter 104 includes an electrode tip 108 at a distal end thereof. Electrode tip 108 may include one or more high-voltage tip electrodes 110 each electrically connected to pulse generator system 102 by a conductor 112. As represented in FIG. 5, and as understood by persons of ordinary skill in the art, steerable catheter 104 may be inserted through blood vessels of a patient to position electrode tip 108 at a desired location proximal to target cells or tissue to be ablated by PFA. By way of non-limiting example, FIG. 5 shows electrode tip 108 guided endovascularly to the intracardiac space of a patient for PFA. Return patches 106A and 106B are separate from catheter 104. Consequently, return patches 106A, 106B may be positioned independently of catheter 104 at respective locations spaced apart from electrode tip 108. As may be understood by additional reference to FIGS. 15 and 16, each return patch 106A, 106B includes a respective electrode plate 114 electrically connected to pulse generator system 102 by a conductor 116. Return patches 106A, 106B are configured and intended to be positioned at respective locations relative to electrode tip 108 so as to form an extended dipole from electrode tip 108 when a voltage difference is applied across the one or more high-voltage electrodes 110 of electrode tip 108 on the one hand, and the electrode plates (return field patch electrodes) 114 of return patches 106A, 106B on the other. When a microsecond-scaled pulse voltage is applied between electrode tip 108 and return patches 106A, 106B, locally isotropic radial electric fields capable of PFA are emitted from the electrode tip 108 to target tissues within a target depth of interest, for example at a depth of about 1-2 cm from the electrode tip. The field is emitted by the stable charge on the capacitor elements rather than the flow of current between them. The depth may be adjusted to suit the needs of a specific clinical case based on the applied voltage V given by V-Q/C, where voltage V is measured in Volts, Q is the plate charge measured in Coulombs, and C is the capacitance in Farads.

In an embodiment, variable patch placement and impedance setting can be performed. Use of four patches can allow variable placement of return energy patches. Patches can be placed to favor a general body region (e.g., around a tumor focus) or with preferential directing to a lateral or posterior versus anterior. In addition, the application of a variable resistor to the collecting terminal of each individual patch can allow tuning of the impedance of the individual patches. This can tune a general strength preference toward or away from a direction (e.g., favoring anterior or posterior ablation during different parts of a procedure). For example, the general strength preference can be adjusted for the posterior versus anterior pulmonary vein anta during a pulmonary vein isolation type atrial fibrillation (AFib) ablation.

As shown in FIG. 5, the system can concentrate a high-intensity field at the tip for targeted ablation while attenuating the return field energy to non-ablative strengths at the patches. Field lines can be distributed across a surface area of the patches. This can dilute the energy over a greater area and reduce energy density and ablation.

In an embodiment of the present disclosure shown in FIG. 8, pulse generator system 102 may include a standard voltage source 120, a waveform generator 122 connected to voltage source 120, and a center tap transformer 124 connected to waveform generator 122. A zero to high positive voltage difference between lines respectively going to patches 106A, 106B and electrode tip 108 is conditioned to a square wave by waveform generator 122 and fed through center-tap transformer 124 to provide equal and opposite polarities to the patches and electrode tip. By way of non-limiting example, the voltage differential may be from approximately −500 V to +500 V up to −1500 V to +1500 V. In an instance, the voltage differential may be from approximately −1000 V to +1000 V.

In an alternative embodiment, separate pulse generator systems may be provided for return patches 106A, 106B and for electrode tip 108 to achieve a voltage differential with equal and opposite polarities to the patches and electrode tip. The separate pulse generator systems would not require a center tap transformer.

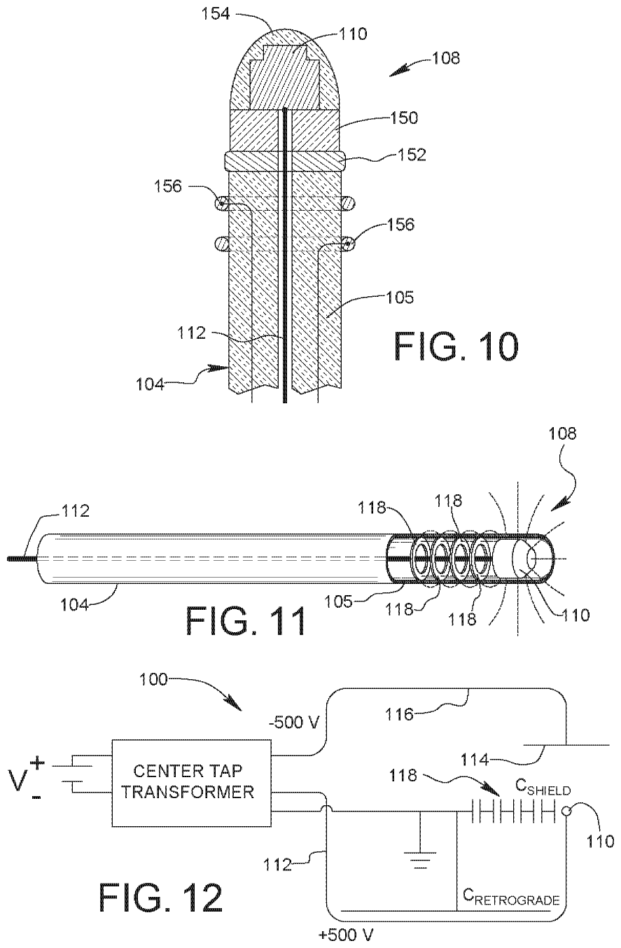

FIG. 10 depicts a configuration of electrode tip 108 at the distal end of catheter 104 in accordance with an embodiment of the present disclosure. A single tip electrode 110 connected to conductor 112 may have a surface layer 150 of dielectric material or other non-conductive or poorly conductive material, such as silicone, polyurethane, polypropylene, polyethylene, fluoropolymers, or mixtures thereof. A coupling 152 may attach non-conductive surface layer 150 to the distal end of catheter 104. Tip electrode 110 may be capped by a layer 154 of electrically insulating material or other non-conductive or poorly conductive material such as silicone, polyurethane, polypropylene, polyethylene, fluoropolymers or mixtures thereof. Catheter 104 may include an electrically insulating outer shell 105 surrounding conductor 112. Outer shell 105 may be made from any of a number of different polymers, for example PELLETHANE®, polypropylene, oriented polypropylene, polyethylene, crystallized polyethylene terephthalate, polyvinyl chloride, etc., in which braiding is embedded. Catheter 104 may be guided or steered by means well-known in the art, such as puller wires. By way of non-limiting example, the diameter of electrode tip 108 may be about 2.4 mm, and the length of electrode tip 108 from coupling 152 to the end of cap layer 154 may be about 3-4 mm. Catheter 104 may further include diagnostic electrodes 156 spaced lengthwise along catheter 104 and exposed outside insulating shell 105 of catheter 104. When the system 100 is not ablating, diagnostic electrodes 156 may be used to measure a voltage difference between the distal tip electrode 110 and each proximal diagnostic electrode 156 as a means of ascertaining the electrical activity of tissue contacted by catheter 104. The electrical signal morphology may indicate what type of cardiac tissue (e.g., atrial, perivalvular, ventricular, venous) is contacted and whether the tissue is “dead ablated” or still “alive”. Proximal diagnostic electrodes 156 may be connected to a voltage measuring device (not shown) that compares measured voltage to ground.

In another embodiment of the present disclosure shown in FIGS. 11 and 12, catheter 104 may include floating elements within the insulating outer shell 105 of catheter 104, for example a series of capacitor rings 118, that shield and guide electric field emissions specifically to the distal electrode tip 108, eliminating potential off-target ablation.

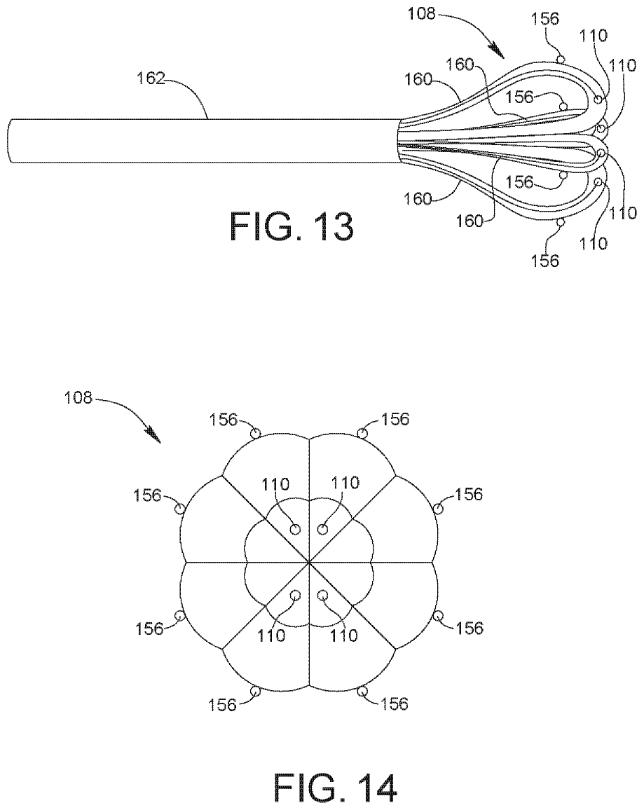

In yet another embodiment of the present disclosure shown in FIGS. 13 and 14, electrode tip 108 may be modified to have an enlarged deformable spheroid configuration compromised of closely spaced and cross-linked linear electrodes 110 with insulation covering 160 and with spaced diagnostic electrodes 156 outside the insulation covering 160. The deformable spheroid configuration of electrode tip 108 may be moved in a compressed spring-loaded state within a delivery sheath 162 at a distal end of catheter 104 to a desired treatment site within the patient, and launched from delivery sheath 162 to resiliently assume the enlarged spheroid configuration for treatment. The configuration of electrode tip 108 shown in FIGS. 13 and 14 may be applied, for example, to pulmonary vein PFA for treating atrial fibrillation.

Additionally, the shape of electrode tip 108 provides cylindrical based symmetry, or variants with near planar surfaces, which vastly reduces the local attenuation of electric fields in space. The configuration of electrode tip 108 is not limited to the configurations depicted herein, and may be adapted to suit specific PFA applications. As another example, electrode tip 108 may be modified to have a wedge shielding configuration applicable to ventricular tachycardia scar homogenization.

In an embodiment of the present disclosure, return patches 106A, 106B may be applied externally to the patient's mid-axillary line of the chest as shown in FIG. 5 so as to avoid blocking access to and viewing of the patient's cardiac region during surgery. In other embodiments, return patches 106A, 106B may be positioned at other locations externally on the patient or internally within the patient. The surface area of each electrode plate 114 may be much greater than the surface area of the one or more tip electrodes 110 concentrated at electrode tip 108, thereby providing a strong local ablative electric field adjacent electrode tip 108 in the region of the target tissue, and an attenuated non-ablative electric field returning to patches 106A, 106B at a location away from the target tissue. This allows the field lines to be distributed across most or an entirety of a surface area of the patches 106A, 106B. A ratio of the surface area of the one or more tip electrodes 110 concentrated at electrode tip 108 to the surface area of each electrode plate 114 may be, for example, on the order of about 1:1,000 or less. With an applied voltage difference supplied by pulse generator system 102, the return patches 106A, 106B cooperate with the one or more tip electrodes 110 concentrated at electrode tip 108 to provide an extended dipole from electrode tip 108 as shown in FIG. 5.

Regarding the ratio of the surface area of the one or more tip electrodes 110 concentrated at electrode tip 108 to the surface area of each electrode plate 114, the surface area of the tip is smaller than that of the patches. In an embodiment, the surface patches may be as wide as possible without obstructing the placement of other monitoring devices on the patient. For example, the focal ablation tip may be approximately 3-4 mm length and 2.5 mm in width for visualization of fluoroscopy and mapping. A wider catheter can still limit surface area of electrodes to maintain this ratio.

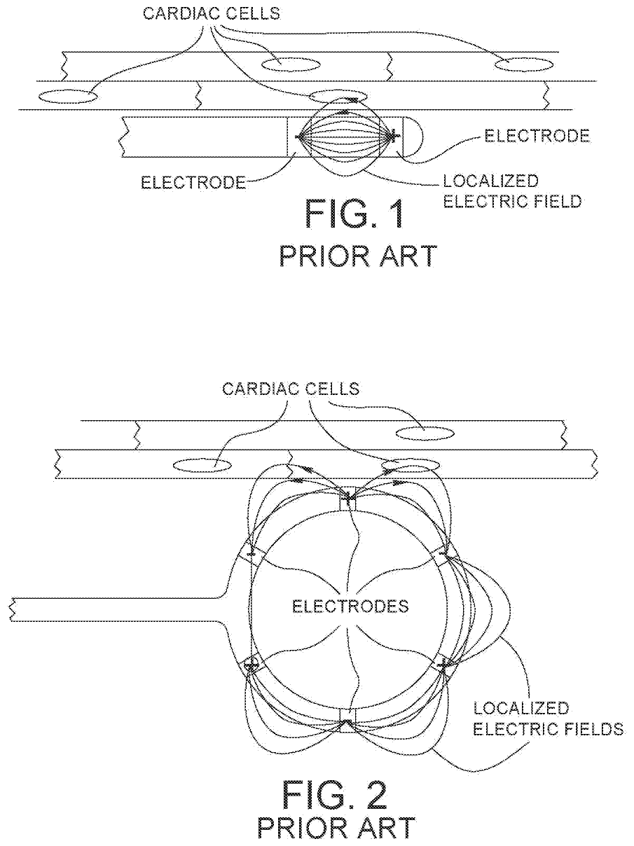

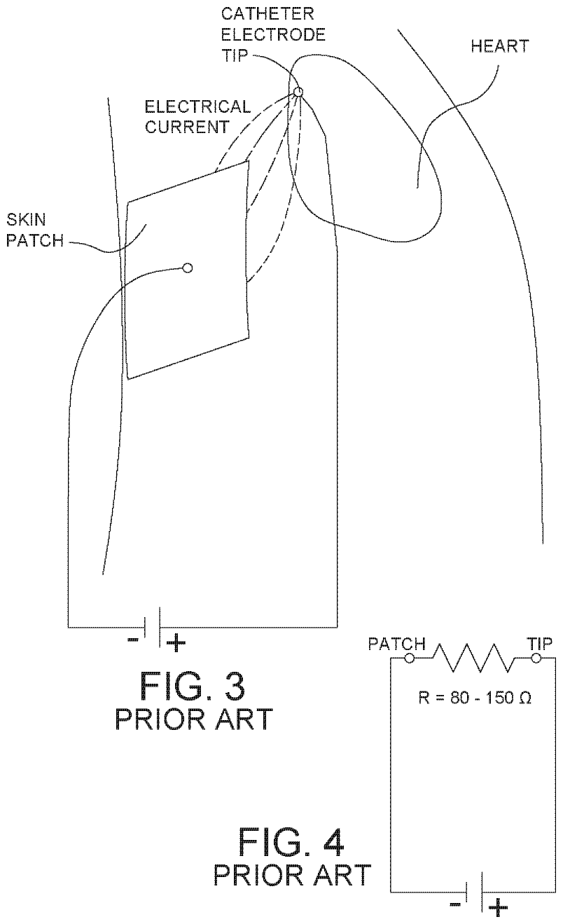

The extended dipole from electrode tip 108 provides local ablative electric fields having substantially radially directed field lines through the target tissue. The radially-directed field lines are best seen in FIG. 6. Advantageously, the radial electric fields are directed predominantly perpendicular to cell membranes regardless of the orientation of electrode tip 108. An endovascular catheter can have the spatial relationship to the long axis of the cardiac cells depicted in FIG. 6. By contrast, previous endovascular PFA systems utilizing a local dipole with close spacing of high and low voltage electrodes, as illustrated in FIGS. 1 and 2, create electric fields having field lines directed substantially parallel to the cell membranes of the target tissue. Fundamental biophysics dictate that only the components of field lies perpendicular to the cell membrane contribute to electroporation. Thus, parallel directed fields emitted by previous systems drastically limit the electroporation efficiency of such systems. In accordance with the present disclosure, electric field lines are radially directed and, consequently, maximize perpendicularity to target cells for improved electroporation efficiency. Thus, FIG. 6 shows that an endovascular endocardiac ablation catheter tip emitting a radially-directed electric field to approach perpendicularity can provide improved results. Embodiments disclosed herein using an extended dipole create such radially-extending electric field geometries in all directions from the catheter tip.

The extended dipole from electrode tip 108 provides an electric field radiating outward from electrode tip 108 with an attenuation factor on the scale of about 1/r to 1/r², rather than the significantly more rapid attenuation on the order of 1/r³ exhibited by localized dipole systems of the prior art. System 100 generates a strong ablative radial electric field at the site of target tissue that attenuates and distributes to non-ablative strength in regions beyond the target tissue approaching return patches 106A, 106B. The slower electric field attenuation factor of system 100 as compared to previous systems allows for the possibility of effective transmural therapy.

An embodiment of return patches 106A, 106B is illustrated in FIGS. 15 and 16. Return patches 106A, 106B, may include high resistance and insulating/dielectric elements covering electrode plate 114 to further limit current delivery to the patient. For example, each return patch 106A, 106B may include an outer shell 130 made of electrically insulating material, and a surface layer 132 covering a surface of electrode plate 114 and interposed between electrode plate 114 and outer shell 130. Surface layer 132 may be made from a dielectric material or other non-conductive or poorly conductive material, such as silicone, polyurethane, polypropylene, polyethylene, fluoropolymers, or mixtures thereof. A predominance of silicone in surface layer 132 is advantageous for flexibility of return patches 106A, 106B. An outer adhesive layer 134 may be provided over a front surface of each return patch 106A, 106B for adhering the return patch to skin of the patient. Electrode plate 114 may be dimensioned as a matter of design choice. By way of non-limiting example, electrode plate 114 may be a rectangular plate measuring approximately 10 cm wide by 20 cm long. As will be understood, return patches 106A, 106B are designed to further contribute to the dielectrically reinforced system of the present disclosure which limits unwanted induced current.

Electrode plate 114 may be formed of an electrically conductive metal, such as stainless steel, copper, aluminum, and nickel-aluminum-bronze alloys. As best seen in FIGS. 16, 17A, and 17B, electrode plate 114 may be configured to reversibly bend along one or more bend lines 136 to approximately conform to the patient, for example to fit the curvature of the lateral chest of a patient. In the illustrated embodiment, two bend lines are provided by aligned intermittent gaps 138 through electrode plate 114 and crimps 139 in electrode plate 114 defining integrally formed hinges in the electrode plate, thereby dividing electrode plate 114 into a central region 140 between a pair of wing regions 142. Only one bend line may be provided, or more than two bend lines may be provided. Outer shell 130, dielectric surface layer 132, and adhesive layer 134 may be flexible to bend with electrode plate 114.

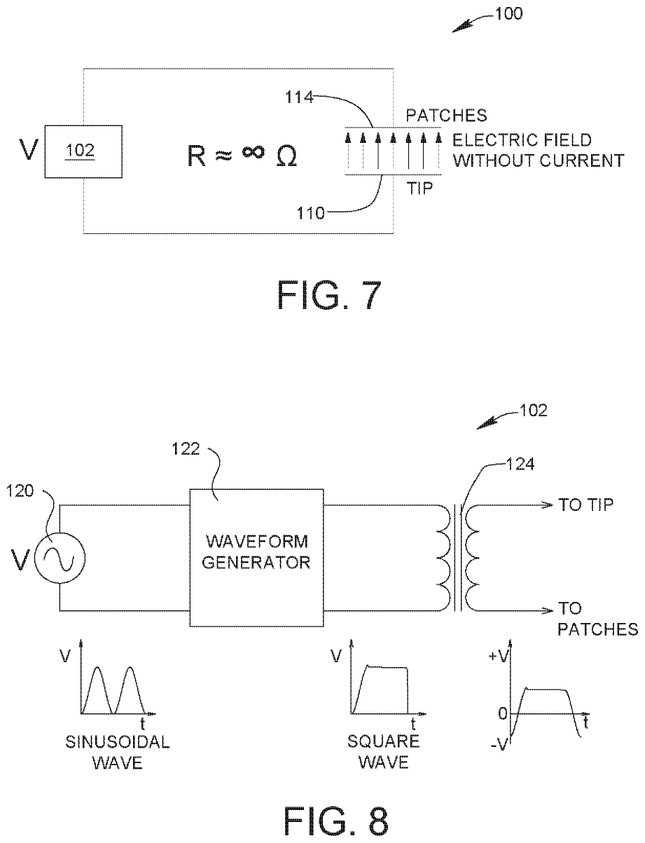

The dielectrically reinforced aspect of system 100, together with the spatially dispersed geometric layout of electrode tip 108 and return patches 106A, 106B, provide a high resistance, high capacitance system that directs emitted fields from the electrode tip 108 with minimal current delivery to the patient. The distance between each tip electrode 110 and each electrode plate 114, which for most patients is in a range of 10 cm to 20 cm, greatly increases the resistance to thermal injury currents compared to the previous systems using closely spaced high-voltage components. Additionally, as mentioned above, high resistance power delivery lines in catheter 104 and dielectric surfacing of return patches 106A, 106B contribute to this beneficial property. Thus, as illustrated by FIG. 7, system 100 essentially treats the patient as a dielectric within a capacitor defined by the insulated electrode tip 108 and insulated patches 106A, 106B. This approach is in contrast to the approach of the prior art, which essentially treats the patient as a resistor conducting a current. Applicant has taken advantage of the realization and understanding that electroporation is driven by metabolic activity of the cells that generates ionic gradients across a cell membrane in the presence of an electric field, and that electric current is not necessary to drive electroporation.

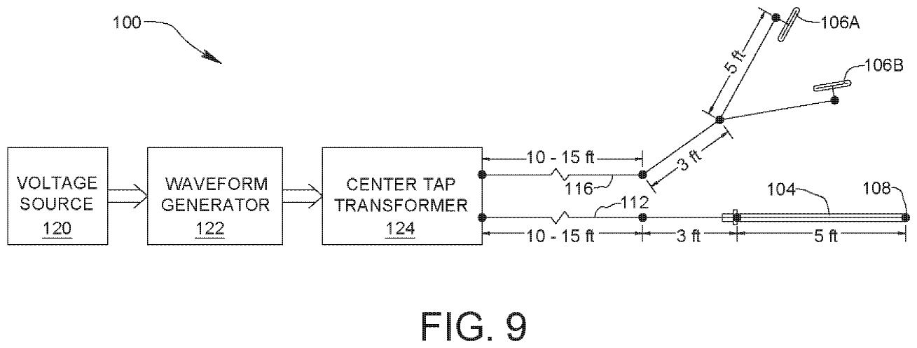

The capacitor of system 100 can reach steady state charge configurations with waveform generators that generate high temporal resolution (20-100 microsecond) square-wave pulses via Fourier summation. For example, in one embodiment, waveform generator 122 may be configured to generate a square wave pulse via Fourier summation of sinusoids to deliver very high temporal resolution pulses. The length of conductor 112 from pulse generator system 102 to electrode tip 108 may be in the range of 18-23 feet (5.486-7.010 meters), and the expected length from electrode tip 108 to return patches 106A, 106B may be approximately in the range of 10 cm to 20 cm. Another example is shown in FIG. 9. These lengths are sufficiently short compared to the wavelengths comprising such high resolution pulses that steady state charge configurations on the capacitor system are obtained during pulse generation to enable system 100 to accommodate all frequency components of high resolution pulse durations within a 10-100 microsecond range. As a result, PFA catheter system 100 may be powered by a variety of pulse generators, and is compatible with multiple power delivery platforms including those with high frequency components for higher temporal resolution. This represents a difference between PFA catheter system 100 and the prior art, further enabling system 100 to be compatible with a variety of power generating systems including those requiring high frequency waveforms.

The geometry of system 100 and location of return patches 106A, 106B away from electrode tip 108 also overcomes the problem of stray and parasitic capacitance associated with delivery of high voltages to closely spaced electrodes. Return patches 106A, 106B are positioned outside the components of ablation catheter 104 and away from the power delivery line (e.g., conductor 112) and tip electrodes 110 within ablation catheter 104. This allows power delivery from pulse generator system 102 to be focused to the ablation electrode tip 108, overcoming stray capacitance.

Similar to localized dipole systems of the prior art, system 100 of the present disclosure may be used to deliver multiple doses of PFA per heart beat (about 60 doses per beat), but with the advantage of stronger, radially-directed electric fields.

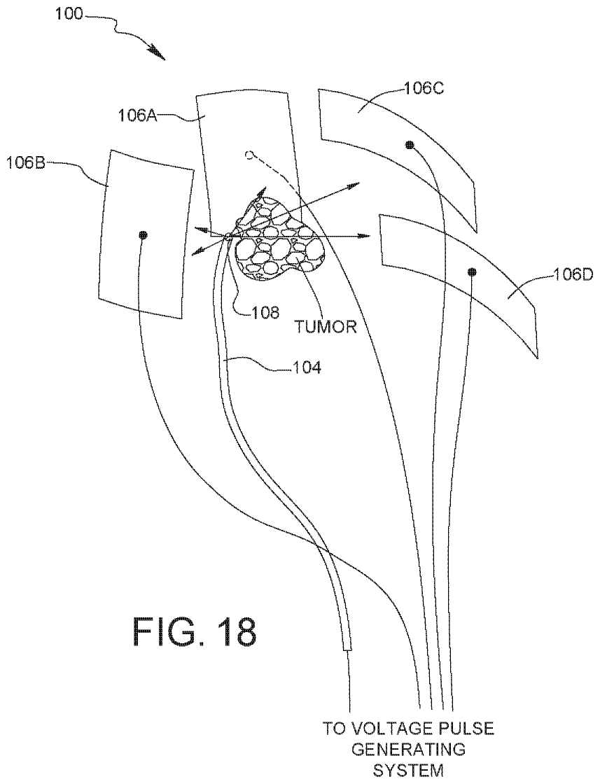

System 100 of the present disclosure is not limited to treating cardiac arrhythmias, and may be adapted for treating other diseases or conditions. For example, system 100 may be adapted for interventional oncology applications. For example, as schematically illustrated in FIG. 18, electrode tip 108 may be designed as an arterial small diameter tip steerable to perivascular tumors for extra-cardiac tumor PFA. System 100 may also be adapted for PFA of non-cardiac tumors by providing additional return patches, as represented by additional return patches 106C and 106D in FIG. 18.

The various embodiments disclosed herein can use both high voltage and low voltage poles. Any electroporation efficiency can be related to the perpendicularity to cell membrane and field strength. The architecture of cardiac cells may require that field extend radially and isotropically from an endocardial tip. A local monopole can generate a strong radially-directed field over long distances, which can optimize perpendicularity of the field relative to cell membranes. Return energy electrodes may be widely-distributed and distant from sensitive tissue. There can be benefits to penetrating tissue boundaries via permittivity (electroquasistatic) versus purely conductive current based methods. Extended currents can follow unpredictable paths while local dipole currents may short therapy. There also can be dynamic tissue effects on specific tissue architecture. Simulations predict that radially-extended geometries can have an iterative depth effect with high conductance ablated zones.

A patient can be modelled as a resistor in a high-voltage battery applied for a short pulse. However, an objective can be to produce a TMV via electric fields not necessarily current. Capacitors can store electric fields with electrostatic distributions without current. The patient can instead be modelled more as a dielectric in a capacitor rather than a resistor. This can be mimicked by dielectrically re-enforcing a tip and patch electrodes with insulating material. However, the generation of a field across our tip to patch capacitor may be reduced by the conductive properties of biological tissue where interstitial ions can flow to almost immediately counter this field. Time-varying voltages (i.e., electroquasistatic) can generate currents across the dielectric space of the capacitor via displacement currents. The varying electric field and displacement can sustain a TMV while minimizing the need for large direct current.

In standard AC circuit theory, as the frequency of the voltage source is increased, then the impedance of capacitor resistor circuit becomes that of a conductor with the field in the conductor carrying displacement rather than direct current. The displacement current can jump the insulative gap.

Using EQS field generation, the electrodes can be reinforced with resistive material to reduce direct current delivery into tissues and so the field jumps this gap. The ability for a field to penetrate resistive material that limits direct current can be directly proportional to the frequency of the applied voltage (i.e., the displacement current jumping the gap). At low enough frequencies (e.g., <1-5 MHz), a time-varying electric field can produce an equivalent TMV change as a constant electric field while enabling the electrodes to be dielectrically re-enforced and direct currents to be reduced.

Referring to FIG. 19, the dimension (e.g., tip to patch distances) of embodiments disclosed herein can be consistent with an EQS approximation to the field and voltage interdependence. In an instance, the EQS approximation may be valid if the product of frequency and length divided by c is less than 1 (e.g., < 1/10). For example, a 1 MHz pulse may be valid up to 30 meters. This may be slightly reduced in tissue by multiplying by tissue permittivity or the square root of the tissue permittivity. In such an instance, voltages are essentially in phase throughout. For example, a 1 MHz pulse is valid up to dimensions of 2.34 meters, which is larger than average tip to axilla distance.

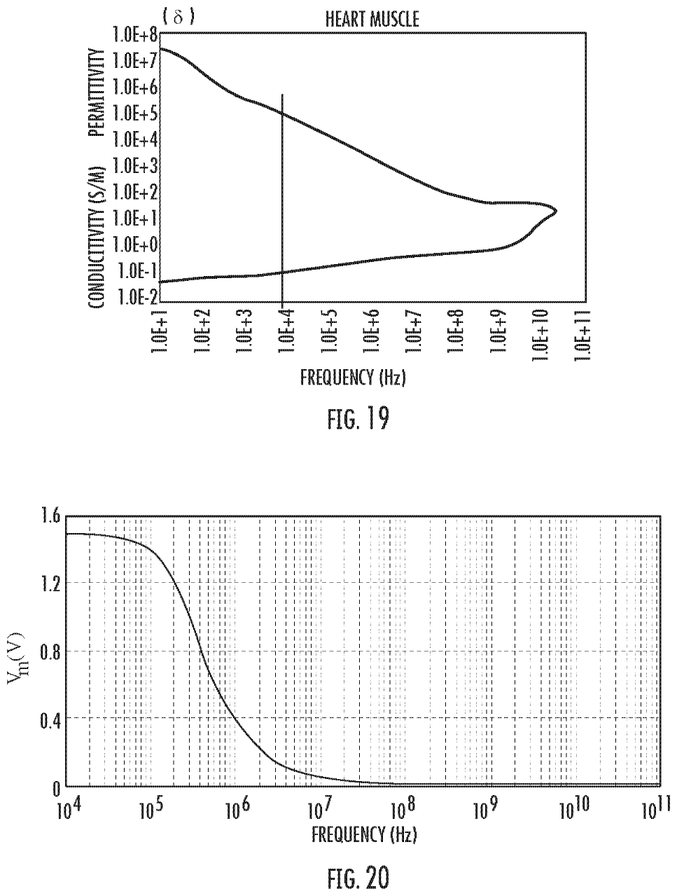

FIG. 20 shows that some of this EQS energy may not convert to entirely TMV at higher frequencies. Some energy may convert to oscillating ion flows instead. Thus, less than 1 MHz frequencies may be used, though other frequencies are possible if the oscillating ion flows are addressed. These less than 1 MHz frequencies may not penetrate all insulator electrode coatings, but can penetrate coatings that are in low conductance/semi-conductance range. In an instance, the frequencies may be from 250 kHz to 300 kHz.

Sinusoidal pulse alone may constitute an approximate reduction in current of 36% (area under curve of sinusoid versus square wave) compared to a square-wave electrocurrent pulse of previous systems. The resistance of the chest wall is roughly 100 ohms using standard defibrillation patches (e.g., with conductive gel). Additionally, the disclosed tip geometry can create an approximate 4 mm×3.14×2.5 mm cross sectional area (0.0000314 m²). Resistance in ohms can be calculated as a 0.3 mm coating of silicon of 20,000 ohms versus a 0.3 mm coating of germanium of 4.4 ohms. In this calculation, the resistivity of graphite is 0.00005 ohm m, germanium is 0.46 ohm m, and silicon is 2300 ohm m. The current can be reduced by 4% for a 0.3 thick germanium coating. Current output can be reduced by more than 99% with silicon.

The thickness and resistance of a coating can result in an approximately <100 kHz reduction because most of field energy may go into TMV charging and IRE. A 250 kHz mild reduction may still mean that all TMV is capable of IRE. A 500 kHz TMV at 0.8 V may be at a lower threshold of IRE in certain instances.

In an instance, the voltage may be from +1000 to −1000 and the frequency may be at <250 kHz based on the peak frequency at which the field would translate into a meaningful TMV for electroporation effect. Rate of electroporation are approximately e^{(TMV}^{²}⁾. The TMV may be >300-500 m Volts.

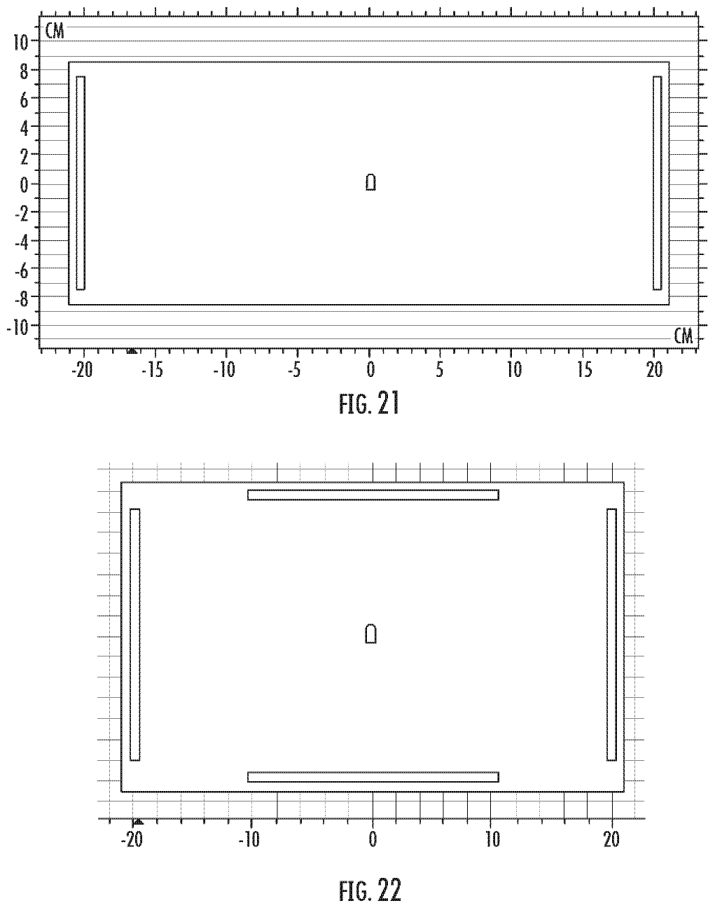

FIGS. 21 and 22 show a layout of two variations of an extended dipole system. FIG. 21 shows a local monopole geometric variation with two negative pole patches at 20 cm separation. FIG. 22 shows an extended dipole (local monopole) with CT overlap. In both variations, the body surface patches serve as opposite electrodes to the central catheter tip that is directed to deliver therapy, which can create an extended dipole. In contrast, simulations using FIG. 21 can demonstrate limited kill zone directionality.

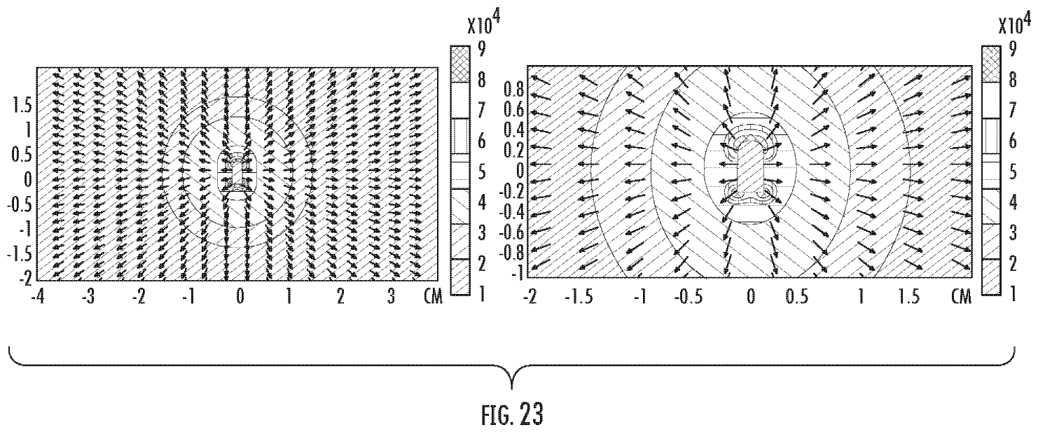

FIG. 23 shows that an extended dipole can create a local monopole with radially-directed fields from a central catheter tip. Using simulations, a +500 V pulse was applied to the tip and a −500 V pulse was applied to the body surface patches. Local to the tip, the radially-directed electric fields extend in all directions. This is a locally-monopole electric field emission. Some of the field is strong enough (e.g., ≥4×10\hat{ }4 V/m) to ablate cardiac tissue if directed against tissue architecture.

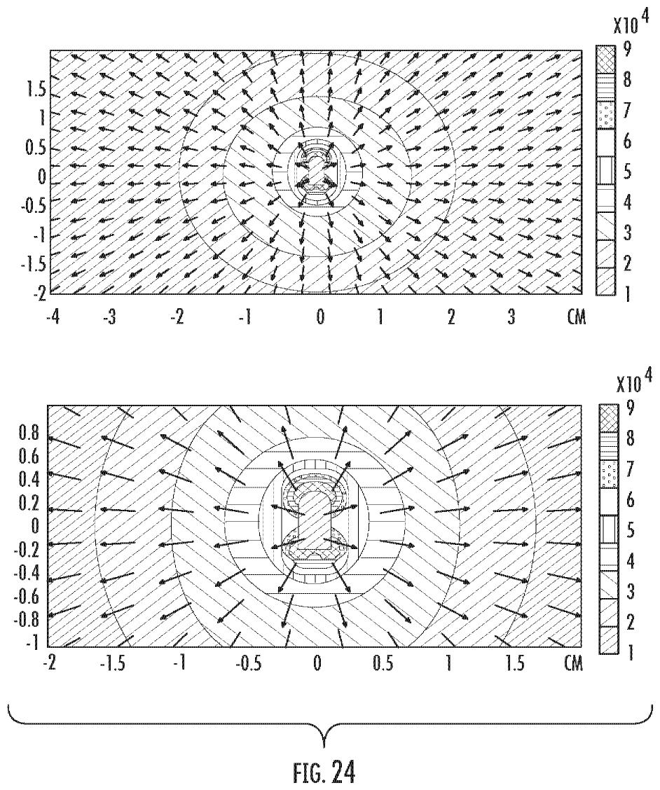

FIG. 24 uses the four patch system of FIG. 22. A near 5 mm single dose kill zone is demonstrated with radial electric fields in all directions. This used voltages from −500 V to +500 V. A 5 mm single dose kill zone is demonstrated in FIG. 24.

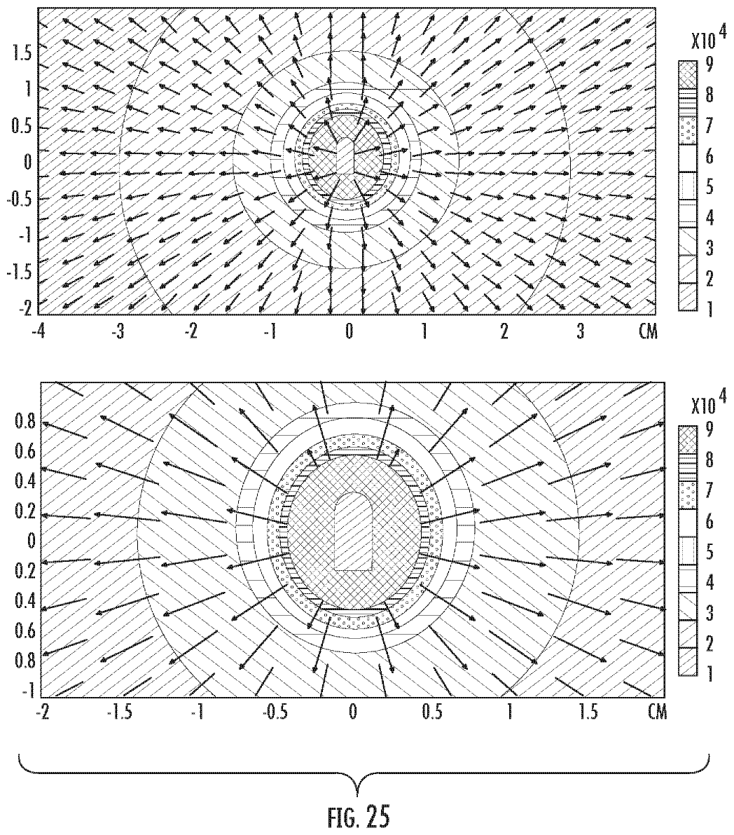

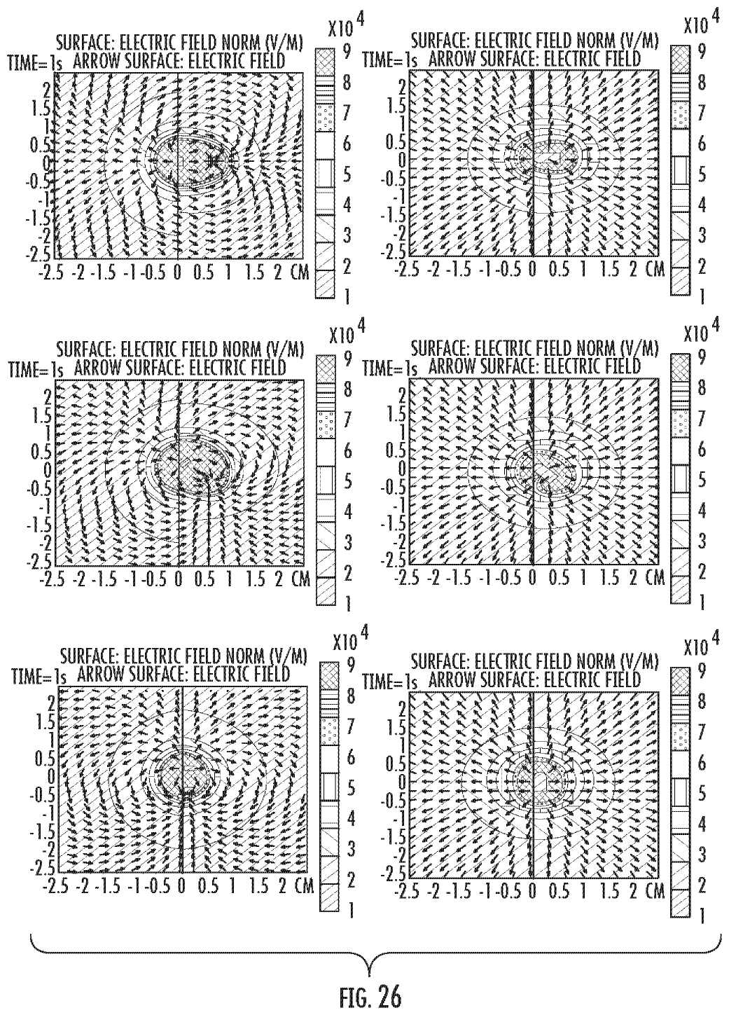

FIG. 25 again uses the four patch system of FIG. 22. At −1000 V to +1000 V, a 1 cm single dose kill zone can be achieved. This kill zone can change when tissue is added because tissue attenuates the depth of energy applied.

Simulations were performed in a saline and skeletal muscle interface. These simulations reflect placement of a catheter tip along the endocardial surface of target cardiac tissue. Tissue simulation is based on the assignment of electrical properties (e.g., relative permittivity and conductance). Skeletal muscle was used because experimental data on its conductive properties follows application of IRE. In addition, skeletal muscle has similar baseline permittivity and conductance to cardiac muscle tissue. Although it may not exactly match in-vivo cardiac tissue, the in-silico set up was uniform for comparison of different systems and configurations.

FIG. 26 illustrates the orientation of the tip with respect to isotropy and ease of use. In terms of pure field direction, embodiments disclosed herein can deliver uniform directionality of vectors across the interface boundary regardless of tip orientation, as shown in the various diagrams of FIG. 26. In terms of energetics, the tissue-saline interface can introduce an energetic barrier, which the disclosed system can more uniformly breach as compared to previous electrode pairs. For example, a single local dipoles pair cannot deliver any IRE capable field across the energetic barrier in the 90 degree lie.

Radial fields can allow the ablated/non-ablated tissue interface to extend the effect of the tip. Tissue ablated from a dose of electric field may become pore-laden and, thus, more conductive. Additional doses of applied field can create iterative depth if the direction of the applied field directs tissue ions toward the interface (e.g., radial-directed). Homogeneity of strength and orientation regardless tissue tip orientation can lead to ease of use.

Although shading in FIG. 26 demonstrates tissue penetration, there is marked anisotropy of field direction based on tip to tissue orientation with the dipole design.

All orientations of the pseudomonopolar (tip-patch) design can have radial emitting field into the tissue regardless of direction with a contiguous section of perpendicular field lines. A dipole may only achieves similar radial/perpendicular field in the 90 degree orientation.

Additionally, locations of parallel field in the dipole mixed between areas of perpendicular field can generate lesion gaps, which can be pro-arrhythmic.

The electrocurrent energy delivery method disclosed herein can manage cross-tissue interfaces. As demonstrated by dipole design there is marked heterogeneity in directional components with this geometry. The 45 degree diagram in FIG. 26 shows that if there is heterogeneity in tissue properties (e.g., in this figure having differing amounts of different materials between electrodes), fields can have marked discontinuities, such as the sworls in field lines and discontinuities in strength shown in FIG. 26. These can reflect less stable penetration pattern with electrocurrent versus the EQS energy delivery.

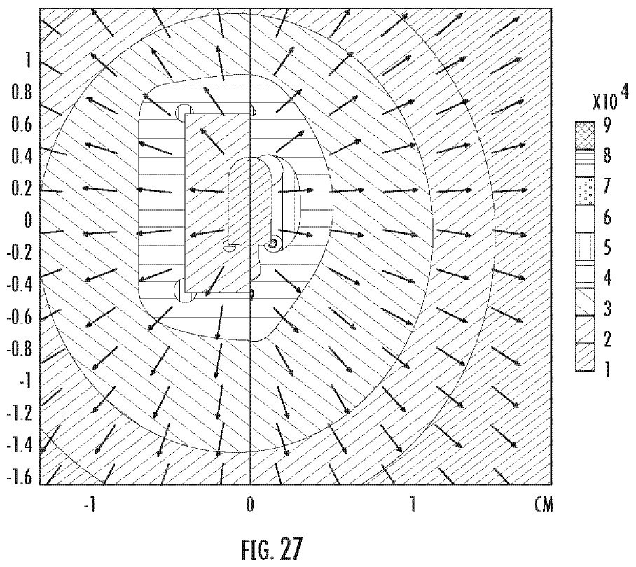

FIG. 27 shows a radial-directed fields simulation. The disclosed system was applied to a muscle/saline interface. Muscle is on the left and saline is on the right. Experimental values of muscle conductivity to applied voltages were used for both baseline and permeabolized states. A rectangular section was given conductivity values of permeabolized (i.e., ablated) tissue.

Note the leftward tissue-tip extension of lighter “kill-zone” field strength beyond that of the kill-zone field depth in the saline. To achieve this interface effect, the emitted field of the system can point toward the interface with an optimized radial field distribution.

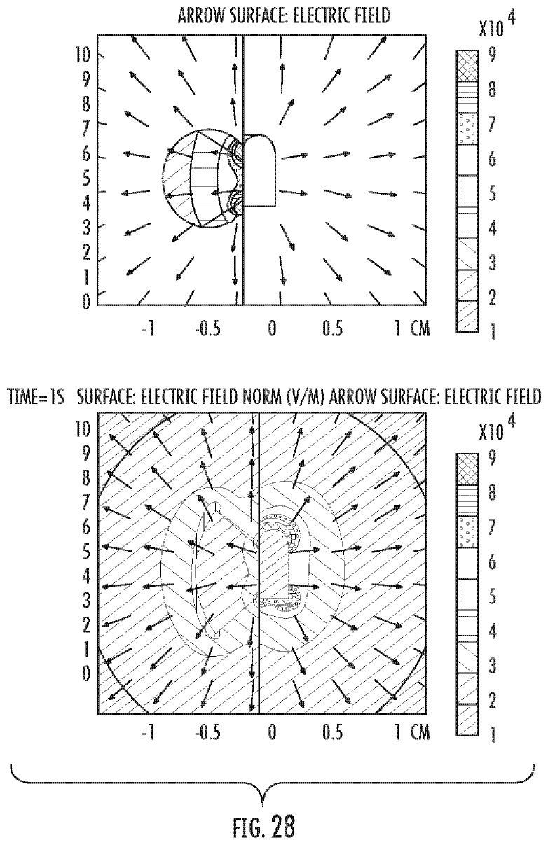

FIG. 28 shows a gap-free lesion extension. This is an example of a two dose sequential lesion growth from the embodiments disclosed herein. The top image in FIG. 28 shows from the first dose application of field, the black lines enclose area of tissue where both the electric field strength and direction meet criteria for ablation/electroporation. The bottom image in FIG. 28 shows a repeated application of electric field with an area of altered tissue with higher conductance. The altered tissue has tip extension effect previously discussed with the lighter region delineating a new region of tissue meeting ablation criteria.

The total ablation zone is illustrated with the arc extending to the left at 5×10⁴ on the bottom image of FIG. 28. Previous systems included a gap due to parallel-directed fields around the total ablation zone. Embodiments disclosed herein may lack such gaps.

Ablation gaps can create an isthmus of conduction that either fails to achieve a primary therapeutic endpoint or are a cause of secondary postoperative arrhythmias (e.g., atypical atrial flutters following standard pulmonary vein isolation (PVI) ablation).

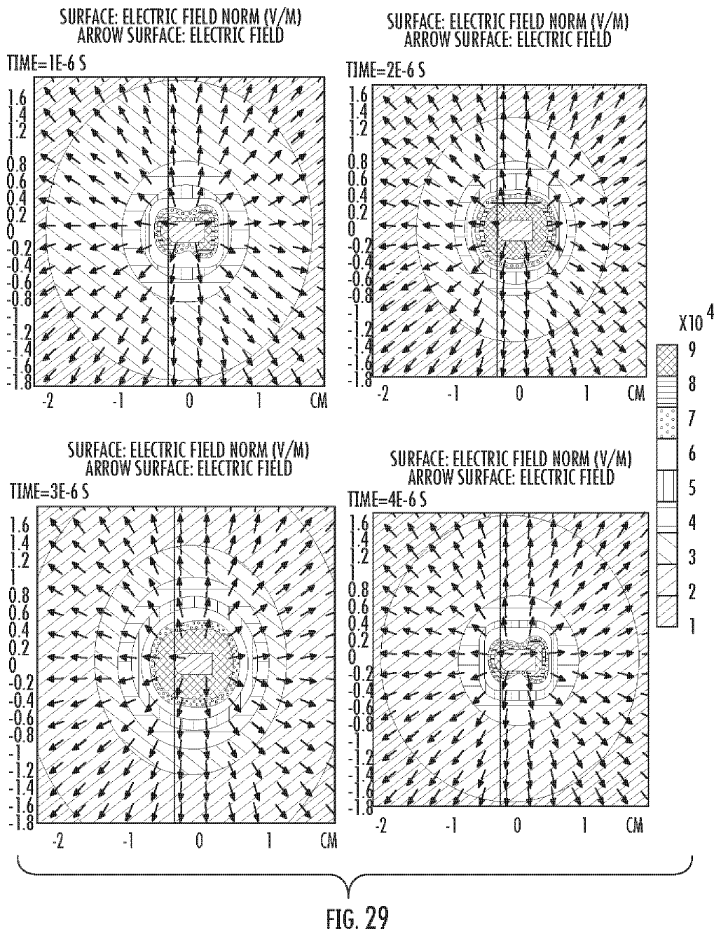

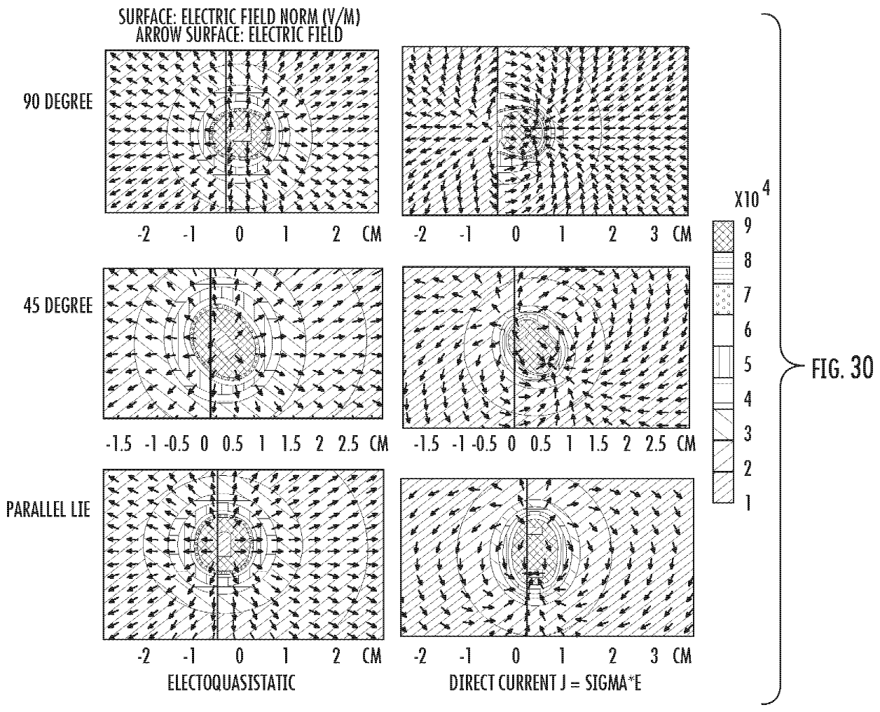

FIG. 29 show a simulation from 1-4 microseconds, with each chart representing a 1 microsecond snapshot during this period. This simulation used graphite-insulation at 1000 V, 100 kHz, and a 90 degree tip. The electric field had a quasi-static distribution with graphite insulation.

The graphite can provide approximately 10-100 relative reduction in conductivity when coating a standard tip electrode material. This also can provide a quality radial field, such as with IRE levels held to >½ cm on initial application throughout >90% duty cycle. Permittivity-based distribution through tissue boundary improved tip orientation independence for operator ease of use.

FIG. 30 shows isotropy for various orientations. As shown in FIG. 30, the same tissue penetration can be maintained with a reduction in direct current. Direct current can cause joule heating. Also as shown in FIG. 30, there is a heterogeneity of field throughout areas of heterogeneous material. This is particularly visible in 45 degree simulations with differing material between the electrode and tissue interface. In whole heart models there is marked heterogeneity of material properties that can accentuate irregular unpredictable field patterns with electrocurrent delivery, which can lead to possible unintended delivery to off-target tissue. Irregular unpredictable field patterns can be avoided with EQS delivery.

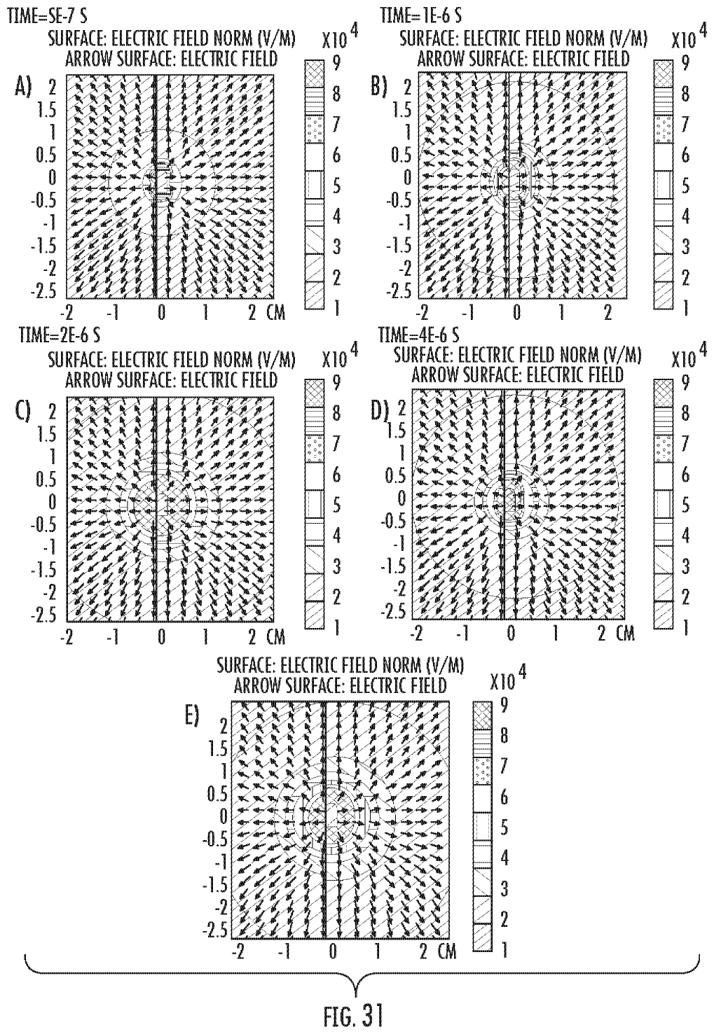

FIG. 31 illustrates time-dependence relationships for embodiments disclosed herein. Diagrams A-D in FIG. 31 are at various time points (0,1,2-3,4 microseconds) of a 100 kHz frequency. This reflects half the wavelength at (5,6,7-8,9) would be equivalent strength in opposite directions. This still provides an ablating field of |+/−400|V/cm. Diagram E in FIG. 31 gives a single readout at peak of complex sinusoid. In this example, it is 100 kHz equivalent to readout at approximately 2-3 microseconds.

For diagrams A-D in FIG. 31, an efficient IRE zone at depths where >80% of duty cycle have |E-Field|≥400 V/cm can be estimated noting that the IRE threshold cardiac tissue includes the positive or negative side of the sinusoid. This is equivalent to IRE reach at (t=⅕× period), such as at 1 microsecond. An equivalent estimate on frequency map is between 5 and 6.

For diagram E in FIG. 31, a 100 kHz equivalent to readout is at approximately 2-3 microseconds. The IRE kill zone at border between 5 and 6 is when >80% of duty cycle is an IRE field.

While particular voltages are used in this example, different voltage peaks are possible. For example, the voltage peak may be from +1500 V to −1500 V.

Embodiments disclosed herein can use a waveform. Example waveforms include a biphasic square wave with interpulse delay, a biphasic square wave, a monophasic square wave, a sine wave, a triangle wave, a sawtooth wave, or other waveforms. Each waveform can be suited for particular applications. Sinusoids can provide interpulse delay. Sawtooth may help bias transmembrane charging. Triangle waveforms may optimize IRE because time varying fields do not require interpulse delay to limit muscle contractions. Of course, other applications, benefits, or waveform configurations are possible.

Higher frequencies may achieve more depth of field, but may need slightly higher peak voltage to achieve same transmembrane potential for IRE. For example, 100-500 kHz can be applied over a 50-100 microsecond length to avoid muscle chronaxie. The IRE dose can be based on derivative of pulse waveforms rather than a peak. A slope of a waveform can determine magnitude of a displacement current EQS term.

An EQS field simulation was performed with various coating materials, coating thicknesses, and frequencies. Coating materials included graphite, germanium, silicon, and high-dielectric ceramic. The dielectric values of the ceramic can be affected by doping, such as with aluminum, which results in a relative permittivity of 200-3500. The coating thickness varied from 0.1-0.5 mm (e.g., 0.2 mm) on, for example, a 2.3-2.5 mm diameter. The frequency varied from 25-500 kHz. These materials and values are merely exemplary and other materials and configurations are possible. The simulations estimate an initial lesion without estimating tissue-tip extension effect or iterative lesion growth.

In an instance, voltage can be reduced using a biphasic sinusoid voltage. Sinusoidal pulse alone compared to a square-wave electrocurrent pulse of a previous system constitutes an approximate reduction in current of 36% (i.e., area under curve of sinusoid versus a square wave).

Current also can be reduced by selecting a dielectric coating. Coatings can act as a resistor, which can optimize the resulting field. The patient can act as a capacitor. The thickness and resistance of the coating can affect the voltage. Graphite, germanium, and silicon all have some conductance, but certain ceramics can be fully insulating. Examples are provided below. These examples are not meant to be limiting and other thicknesses, voltages, and other variables are possible.

In an example, the coating is silicon with a 0.1 mm thickness at 100 kHz (+/−1000 Volts). The estimated initial lesion effective field is 0.5 cm at >80% of duty cycle. IRE level fields for 80% duty cycle are approximately 0.4-0.5 cm. This results in a current reduction of greater than approximately 98% (approximately 5000 ohms). This presents a workable initial dosage tissue penetration with reduced current.

However, a 0.5 mm silicon coating at +/−1000 Volts at 25 kHz and 500 kHz develops little depth of an IRE capable field even at maximal IRE frequencies. The overall resistance may be too high while the dielectric properties are too minimal at this thickness compared to 0.1 mm to give efficient field penetration.

In another example, the coating is graphite with a 0.5 mm thickness at +/−1000 V and a frequency of 25 kHz and 250 kHz. Graphite has relatively high conductance (e.g., 2×10⁵). Compared to heart tissue, graphite will provide high tissue maximal frequency penetrance at even low frequencies (e.g., 25 kHz). This offers some minimal resistance. A reduction in workable frequencies and a 36% current reduction via sinusoid voltage waveform are provided using graphite.

In another example, the coating is germanium with a 0.5 mm thickness at +/−1000 V, and a frequency of 25 kHz and 250 kHz. Germanium offers more conductivity reduction, but still enough to allow maximum frequency penetration at low frequencies (e.g., 25 kHz). There is a good dielectric breakdown field at 10\hat{ }7 V/m, which can maintain dielectric properties at applied fields versus graphite at 10\hat{ }5 V/m.

In another example, the coating is a ceramic. Ceramics can provide maximum resistance and current reduction with desired frequency tissue penetration (e.g., 0.6 cm initial lesion border) at 250 kHz, although at 500 kHz the TMV may be reduced. This is efficient in both frequency and current reduction.

Ceramic can provide good initial lesion IRE field penetration for >80% duty cycle at 0.5-0.6 cm. Current reduction can be >99.99%. The voltage distribution can reach 0 voltage by 1 cm from tip.

Germanium and graphite can lower the titanium tip electrode radius and replace the volume with graphite or germanium. The smaller the high conductive tip to the patch size ratio can increase the field emission. Both germanium and graphite offer high tissue penetrance even at low frequencies. Although germanium and graphite offer less resistance, these materials can offer the option of increasing the thickness of the coating and even reducing the focality of the conductive tip electrode thereby increasing field emission. Silicon at 100 kHz and 250 kHz provide good penetration and resistance at 0.1 mm thickness. Ceramics can provide full permittivity-based distribution at 250 kHz.

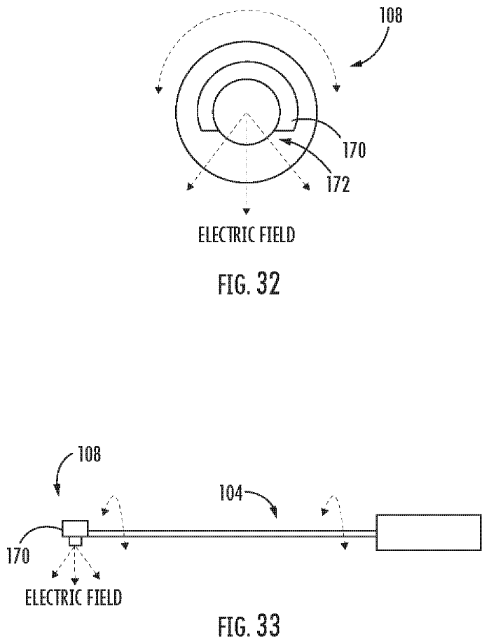

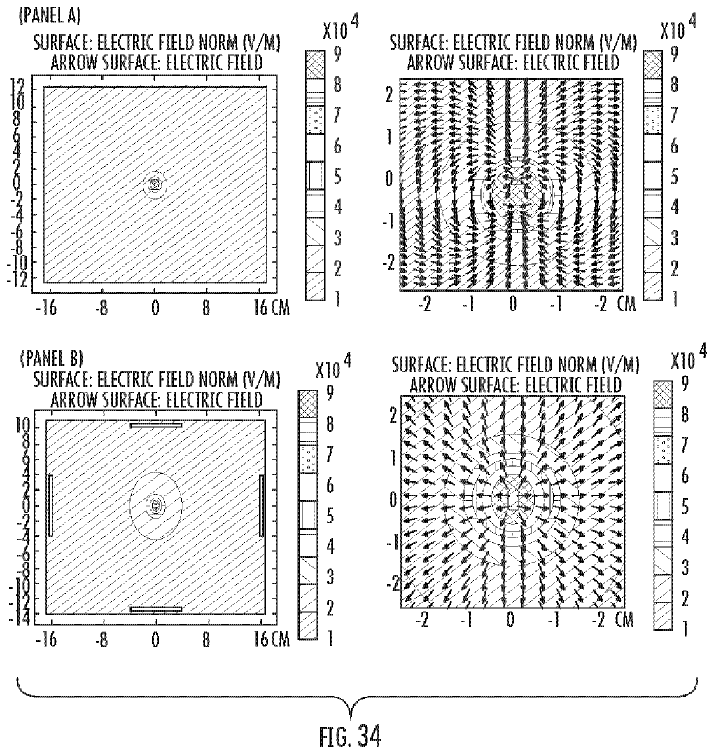

An embodiment is shown in FIGS. 32-33. The electrode tip 108 is at a distal end of a catheter 104. A direction of the electric field emitted from the electrode tip 108 can be rotated by rotating the catheter 104 (as shown in FIG. 33 and corresponding rotation in FIG. 32). The outer shaft of the catheter 104 may be fabricated of a resistive insulation until the electrode tip 108. A central hole allows passage of the drive line conductor to the electrode tip 108 through the catheter 104 thereby connecting the electrode tip 108 to the generator. The central hole can enable tight but free rotation of the drive line.

A turnable wheel at the handle of the catheter 104 (e.g., insulating plastic) can be attached to the drive line so the wheel can be turned either 180 degree Clockwise or −180 counter clockwise, such as using the puller wires. This in turn will rotate the emitting portion of the tip freely toward a desired direction in a 360 degree Field. This is shown in FIG. 32 that the electric field can be rotated around a central axis. The insulation 170 can block or impede the electric field from being emitted except through an aperture 172 in the insulation 170. Thus, most or all of the electric field is emitted through the aperture 172.

Examples of the various fields and effects are shown in FIGS. 34-41.

In FIG. 34, the top (Panel A) is wide view (right) and close up view (left) local dipole electrode geometry. The bottom (Panel B) is wide view (right) and close up view (left) of pseudomonopole (extended dipole) geometry.

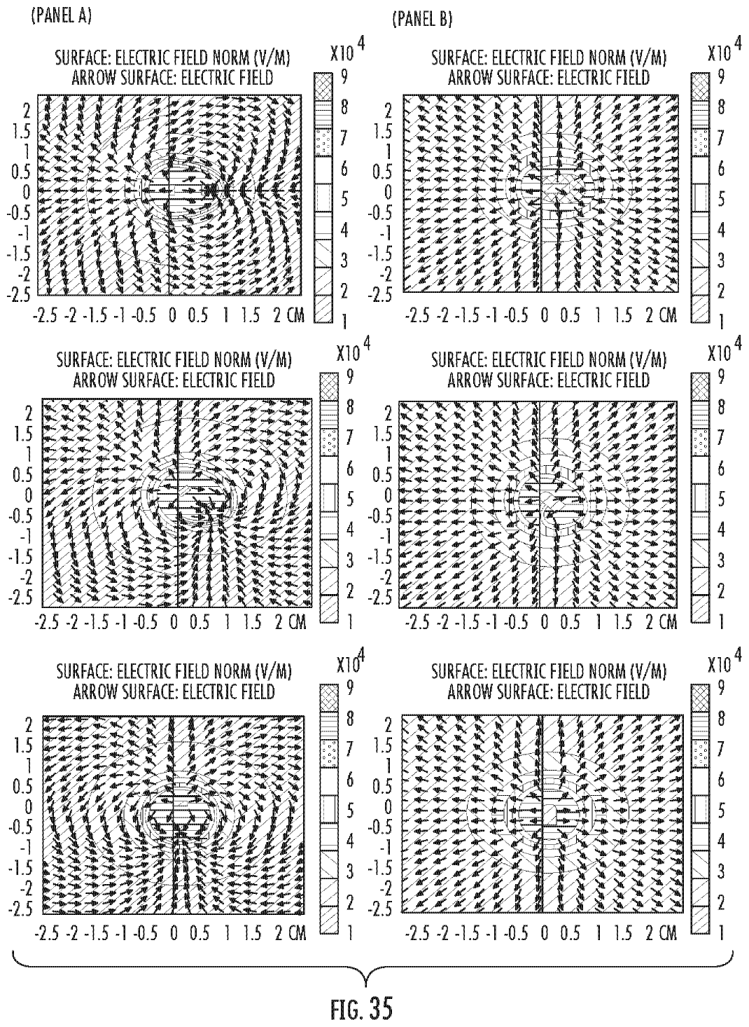

In FIG. 35, the blood lumen/heart tissue interface is a line at x=0. Energy delivery is at +1000/−1000 Volts direct current. The Panel A on the left is local dipole geometry at 90 degree (top), 45 degree (middle), and 0 degree tissue tip orientations. The Panel B on the right is same of the pseudomonopole geometry.

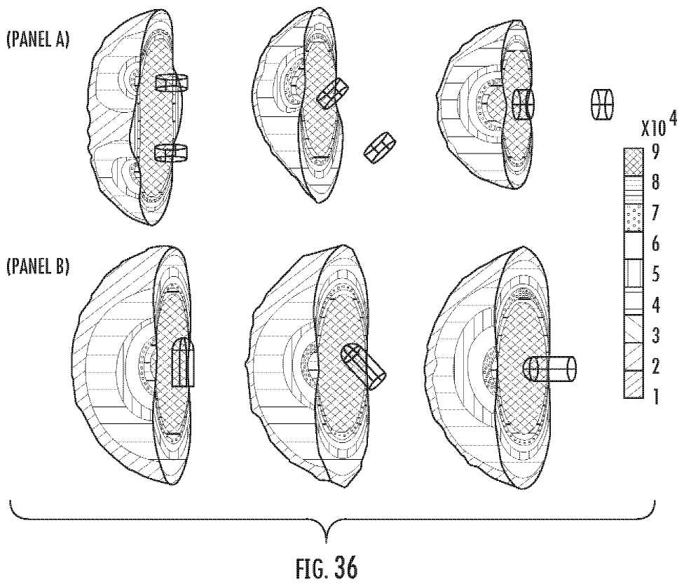

In FIG. 36, the top (Panel A) shows volumes associated with local dipole in 0, 45, and 90 degree orientations respectively. Bottom (Panel B) volumes associated with pseudomonopole in corresponding orientations. The field strength is shown with hatching.

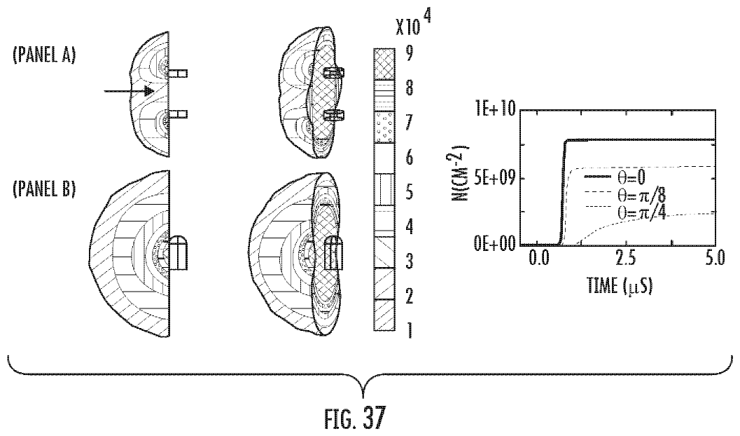

In FIG. 37, Panel A is a local dipole geometry. The black arrow depicts ablation rate gaps region with minimal ablation threshold fields between regions of high ablation fields centered at electrodes. Panel B shows corresponding single dose ablation threshold volumes for a pseudomonopole. The chart on the right depicts variation in pore production rates and peaks at different angles from absolute perpendicular.

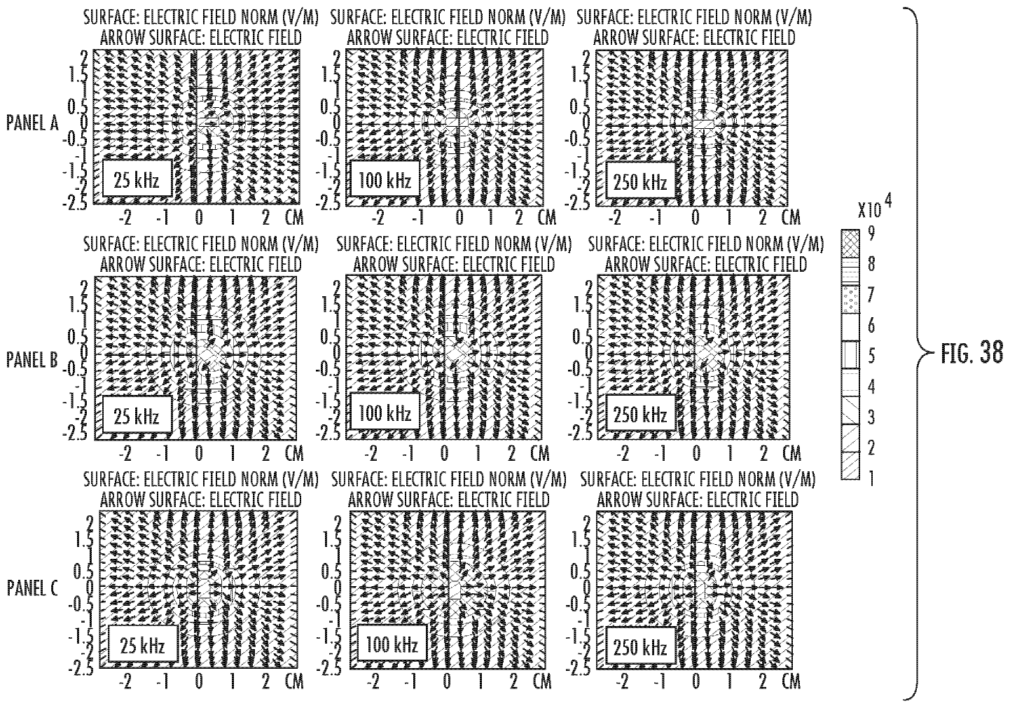

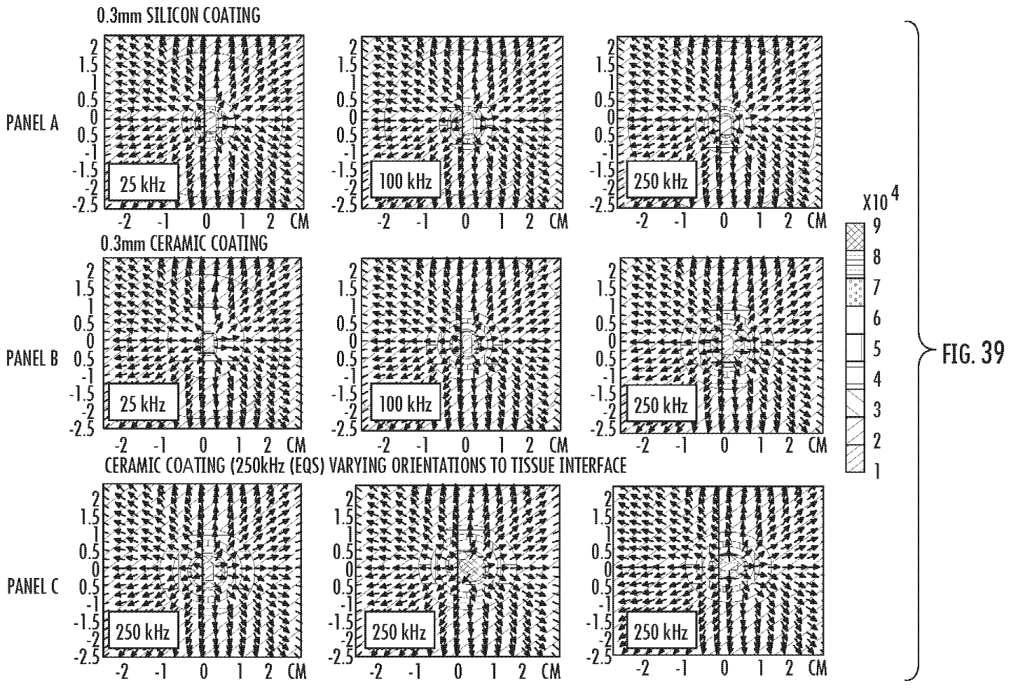

In FIG. 38, the energy protocol is frequency-based AC peak with +1000 to −1000 voltage differentials. A 9×9 display at varying frequency 25 kHz, 100 kHz, and 250 kHz going from left to right with three different tissue/tip orientations at 90 degree, 45 degree, and 0 degree From top to bottom.

In FIG. 39, the top (Panel A) are 0 degree orientation with silicon tip at 25 kHz, 100 kHz, and 250 kHz. The middle (Panel B) are 0 degree orientation with high-permittivity ceramic at 25 kHz, 100 kHz, and 250 kHz. The bottom (Panel C) are 250 kHz simulations of ceramic at three different tissue tip orientations 0, 45, and 90 degree.

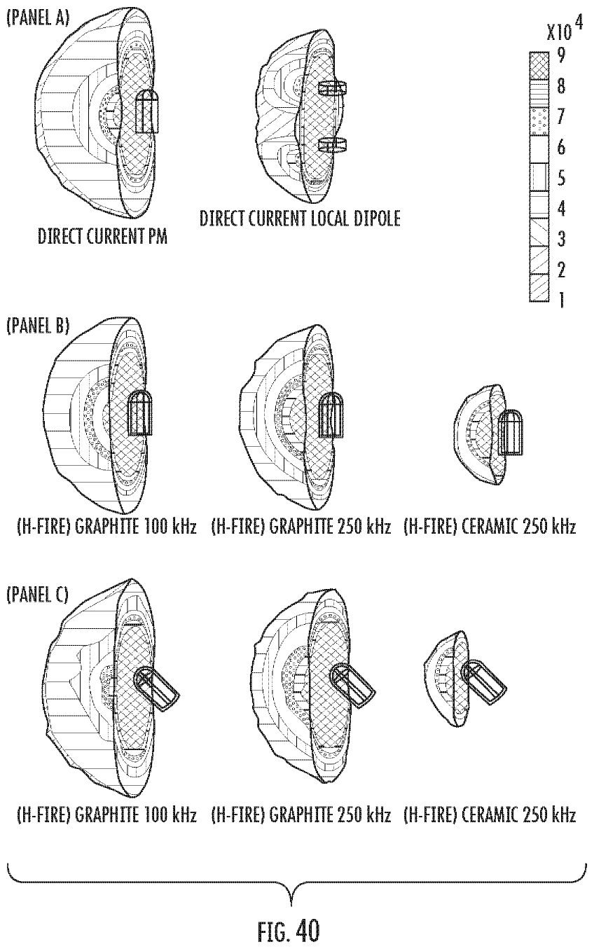

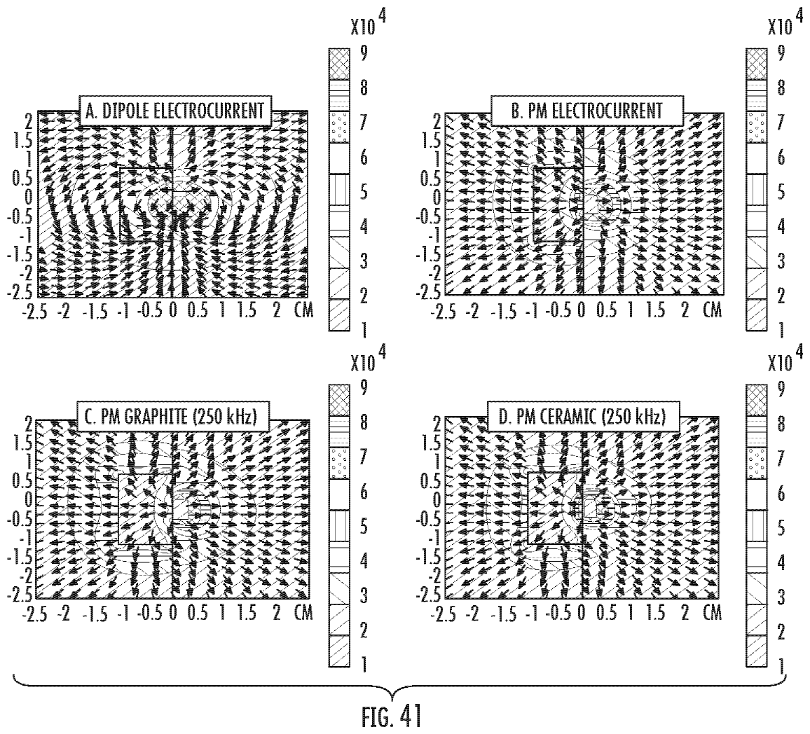

In FIG. 40, the top (Panel A) are direct current volumes of 0 degree orientation pseudomonopole (left) and local dipole (right). The middle (Panel B) are 0 degree high frequency (H-FIRE) simulation of (left) 25 kHz graphite, (middle) 250 kHz graphite, and (right) 250 kHz ceramic. The bottom (Panel C) are 45 degree high frequency (H-FIRE) simulations of (left) 25 kHz graphite, (middle) 250 kHz graphite, and (right) 250 kHz ceramic.

In the simulation of FIG. 41, a 1×2 cm ablated block was simulated. A 0 degree tissue orientation was used throughout. Top (A) is direct current delivery with (left) local dipole. Top (B) is direct current pseudomonopole geometries. Bottom (C) is high frequency (H-FIRE) delivery of graphite coated pseudomonopole at 250 kHz. Bottom (D) is high frequency (H-FIRE) delivery of ceramic-coated pseudomonopole at 250 kHz.

For each of the simulations disclosed herein, the units for field strength can be the same unless specified otherwise.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

1. A tissue ablation system comprising:
a catheter including a distal electrode tip having at least one tip electrode, wherein the at least one tip electrode comprises a non-conductive surface layer;
at least one return electrode positionable independently of the at least one tip electrode, wherein the at least one return electrode comprises a non-conductive surface layer; and
a voltage generator circuit electrically connected to the at least one tip electrode and to the at least one return electrode, wherein the voltage generator circuit is operable to apply a voltage differential across the at least one tip electrode and the at least one return electrode such that the at least one tip electrode is given a polarity opposite a polarity of the at least one return electrode;
whereby tissue located between the at least one tip electrode and the at least one return electrode acts as a capacitive dielectric medium in which an electric field is present and is not dependent on current flow between the at least one tip electrode and the at least one return electrode, and the electric field induces electroporation of tissue cells in a vicinity of the distal electrode tip.

2. The system according to claim 1, wherein the at least one return electrode has a surface area greater than a surface area of the at least one tip electrode.

3. The system according to claim 1, wherein the at least one return electrode is separate from the catheter.

4. The system according to claim 3, wherein the at least one return electrode is embodied in a patch configured for external placement on a skin surface.

5. The system according to claim 1, wherein the voltage generator circuit is an AC voltage generator circuit.

6. The system according to claim 1, wherein the voltage generator circuit is a DC voltage generator circuit.

7. The system according to claim 1, wherein the catheter is a steerable catheter.

8. The system according to claim 1, wherein the electrode tip is configured to be rotatable, and wherein the catheter defines an aperture that the electric field is emitted through.

9. A tissue ablation method comprising the steps of:
positioning a first electrode at a location proximate to a tissue target, wherein the first electrode comprises a non-conductive surface layer;
positioning a second electrode at another location spaced from first electrode and the tissue target, wherein the second electrode comprises a non-conductive surface layer; and
generating a voltage differential across the first electrode and the second electrode such that the first electrode has a positive polarity and the second electrode has a negative polarity;
whereby tissue located between the first electrode and the second electrode acts as a capacitive dielectric medium in which an electric field is present and is not dependent on current flow between the first electrode and the second electrode, and the electric field induces electroporation of tissue cells in the tissue target.

10. The method according to claim 9, wherein the second electrode has a surface area greater than a surface area of the first electrode.

11. The method according to claim 10, wherein the electric field is distributed across most of the surface area of the second electrode.

12. The method according to claim 9, wherein the step of positioning the first electrode comprises guiding the first electrode endovascularly within a patient by means of a catheter.

13. The method according to claim 12, wherein the electric field is primarily emitted through an aperture in the catheter, and further comprising rotating the first electrode in the catheter thereby exposing different areas of the tissue to the electric field through the aperture.

14. The method according to claim 9, wherein the step of positioning the second electrode comprises placing the second electrode against an external skin surface of a patient.

15. The method according to claim 14, wherein more than one of the second electrode is provided, and wherein the second electrodes are positioned at locations around the tissue target on opposite sides of a patient.

16. The method according to claim 9, wherein the electric field extends in all directions from the first electrode.

17. The method according to claim 9, wherein the electric field is a monopole with radially-directed fields from the first electrode.

18. The method according to claim 9, wherein the electric field has radially-extending electric field geometries in all directions from the first electrode.

19. The system according to claim 1, wherein the at least one return electrode includes a high-dielectric ceramic with a relative permittivity from 200 to 3500.

20. The method according to claim 9, wherein a frequency associated with the voltage differential is from 100 kHz to 250 kHz.
